(19) United States
(12) Patent Application Publication
(10) Pub. No.: US 2025/0059579 A1
(43) Pub. Date: Feb. 20, 2025

(54) TEMPERATURE-INDUCIBLE EXPRESSION SYSTEM FOR THERMOPHILIC ORGANISMS

(71) Applicant: DANMARKS TEKNISKE UNIVERSITET, Kongens Lyngby (DK)

(72) Inventors: Sheila Ingemann Jensen, Kongens Lyngby (DK); Vívìënne Henrieke Mol, Kongens Lyngby (DK); Alex Toftgaard Nielsen, Kongens Lyngby (DK)

(21) Appl. No.: 18/720,007

(22) PCT Filed: Dec. 16, 2022
(86) PCT No.: PCT/EP2022/086436

§ 371 Date: Jun. 14, 2024

(30) Foreign Application Priority Data
Dec. 16, 2021 (EP) 21215326.6

(51) Int. Cl. C12P 21/00; C12N 15/75

(52) U.S. Cl. CPC C12P 21/00; C12N 15/75; C12N 2800/101; C12N 2830/003

(57) ABSTRACT

Provided are thermophilic bacteria comprising an expression control system comprising a Tet repressor protein, use of such an expression system to control the expression of a gene of interest in thermophilic bacteria by temperature regulation and methods for producing a gene product of interest in thermophilic bacteria by using this system by temperature regulation.

### FIELD OF THE INVENTION

The present invention relates to thermophilic bacteria comprising an expression control system comprising a Tet repressor protein and use of such a system to control the expression of a gene of interest in thermophilic bacteria by temperature regulation.

### BACKGROUND OF THE INVENTION

In moving towards a more sustainable bioeconomy, ever-increasing interest is shown in fermentative bioproduction as an alternative to petrochemical synthesis. Thermophilic fermentation (>45° C.) has been shown to possess inherent advantages over traditional, mesophilic production (20-45° C.), including, but not limited to, lower risk of contamination, less cooling energy requirement, efficient pretreated-biomass consumption and higher conversion rates. This altogether provides economic incentive to pursue thermophilic production for (bulk) production of chemicals, which is reflected by the increasing research interest in these production hosts. One factor that delays large-scale application of thermophiles is the present toolbox to optimize productivity in target strains.

One commonly used approach to optimize product formation in fermentations is through the induction of the production-related proteins and pathways by the addition of specific inducer compounds. By doing so, one can ensure that there is a separate growth and production phase of the process. In this manner, no carbon is lost to the products in upstream stages of production, where the target is maximal production of biomass. Conversely, induction of a separate production phase leads to increased control over carbon flux towards the products of interest, in turn resulting in higher overall productivities.

The Tet repressor and promoter (TetR-Pₜₑₜ) system is a well-known example of a system which can be used to control the expression of a protein of interest by the addition of an inducer molecule. The Tet repressor (TetR) binds to the tet operon (tetO) sequence present in the tetA promoter and inhibits protein expression from this promoter. Upon addition of the inducer molecule tetracycline or the chemical analog anhydrotetracycline, TetR loses its ability to bind to the tetO sequence, and expression from the tetA promoter can occur. Use of TetR-Pₜₑₜ systems at mesophilic temperatures has been described (Corrigan et al., Plasmid;61:126-129 2008; Kamionka et al., Appl. Environ. Microbiol.; 71:728-733 2005; Wickstrum et al., PloS One;8: e76743 2013; Bertram and Hillen, Microb. Biotechnol.; 1:2-16 2008).

A disadvantage of using externally added inducers is that it leads to an added material cost, as well as introducing an additional risk of contamination upon addition. Finally, specifically for thermophilic fermentations, high temperatures can result in degradation of inducer molecules, which can create heterogeneous populations in the fermentation, being disadvantageous to overall productivity. Therefore, novel systems are needed to allow induction of expression without the need to add expensive, exogenous compounds to the fermentation.

### SUMMARY OF THE INVENTION

It has been found by the present inventors that an expression control system comprising a Tet repressor (TetR) protein and a promoter comprising a tet operator (tetO) sequence can be used to control protein expression in a thermophilic host cell by temperature-regulation at thermophilic temperatures, in contrast to the classical induction of such a system by addition of the inducer molecule tetracycline or derivatives thereof.

So, in a first aspect, the present invention relates to a thermophilic host cell comprising a heterologous expression control system comprising a Tet repressor (TetR) protein and a first promoter comprising a tet operator (tetO) sequence to which the TetR protein can bind, wherein the first promoter is operatively linked to at least one gene of interest.

In a second aspect, the present invention relates to use of an expression control system comprising a TetR protein and a first promoter comprising a tetO sequence to which the TetR protein can bind to control the expression of at least one gene of interest in a thermophilic host cell by temperature regulation.

In some embodiments, the thermophilic host cell further comprises a gene encoding the TetR protein, optionally wherein the gene encoding the TetR protein is operatively linked to a second promoter.

In some embodiments,

(a) when the thermophilic host cell is incubated in a culture medium having a first temperature, the expression of the gene of interest is not induced; (b) when the thermophilic host cell is incubated in a culture medium having a second temperature, the expression of the gene of interest is induced; and (c) the second temperature is at least about 4° C. higher than the first temperature.

In a third aspect, the present invention relates to a method of controlling the expression of at least one gene of interest in a thermophilic host cell comprising an expression control system comprising a TetR protein and a first promoter comprising a tetO sequence, wherein the first promoter is operatively linked to the at least one gene of interest, the method comprising:
(i) incubating the thermophilic host cell in a culture medium having a first temperature; and
(ii) incubating the thermophilic host cell in a culture medium having a second temperature which is at least about 4° C. higher than the first temperature, wherein steps i) and ii) can be conducted in any order,
   wherein the expression of the at least one gene of interest is induced at the second temperature but not at the first temperature, thereby controlling the expression of the at least one gene of interest.

In a fourth aspect, the present invention relates to a method of producing a gene product of interest in a thermophilic host cell comprising an expression control system comprising a TetR protein and a first promoter comprising a tetO sequence to which the TetR protein can bind, wherein the first promoter is operatively linked to a gene of interest encoding the gene product of interest, the method comprising:
(i) incubating the thermophilic host cell in a culture medium having a first temperature; and
(ii) incubating the thermophilic host cell in a culture medium having a second temperature which is at least about 4° C. higher than the first temperature; and
(iii) optionally, isolating the gene product,
   wherein the expression of the gene encoding the gene product of interest is induced at the second temperature but not at the first temperature, thereby producing the gene product of interest.

In some embodiments, the method according to the third or fourth aspect comprises a prior step of transforming or transfecting the thermophilic host cell with one or more nucleic acid constructs comprising the first promoter, the at least one gene of interest and a gene encoding the TetR protein, optionally wherein the gene encoding the TetR protein is operatively linked to a second promoter.

In some embodiments, the medium has not been supplemented with tetracycline or an analog thereof which binds to the TetR protein.

In some embodiments, the first temperature is at most about 60° C., the second temperature is at least about 65° C., or both.

In some embodiments, (a) when the TetR protein is bound to the tetO sequence of the first promoter, the promoter is repressed; and (b) when the TetR protein is not bound to the tetO sequence of the first promoter, the promoter is active.

In some embodiments, the TetR protein
a) comprises an amino acid sequence at least 60% identical to a sequence selected from SEQ ID NOs: 1, 32-56, and 79-82,
b) has a melting temperature between 40° C. and 90° C., preferably between 55° C. and 90° C., more preferably between 60° C. and 90° C.,
c) comprises a helix-turn-helix (HTH) region, which comprises a DNA-binding domain and a sub-region of at least 4 consecutive amino acid residues, which sub-region destabilizes upon temperature increase, optionally wherein the sub-region has a change in root mean square fluctuations (ΔRMSF) score above 1.5 times the average ΔRMSF score for the whole protein in dimeric form when measured by molecular dynamic analysis using the software program CABS-flex 2.0, or
(d) a combination of (a) and (b), (a) and (c) or all of (a) to (c).

In some embodiments, (a) the TetR protein comprises an amino acid sequence at least 60% identical to SEQ ID NO:1; and/or (b) the tetO sequence comprises a nucleotide sequence at least 65% identical to SEQ ID NO: 5.

In some embodiments, the expression control system comprises a TetR protein comprising an amino acid sequence at least 90% identical to SEQ ID NO:1, a tetO nucleotide sequence at least 90% identical to SEQ ID NO:5, a first promoter comprising a nucleotide sequence at least 90% identical to SEQ ID NO: 14, and, optionally, a second promoter comprising a nucleic acid sequence at least 90% identical to SEQ ID NO: 13.

In some embodiments, the thermophilic host cell has an optimum growth temperature in the range of about 55° C. to about 65° C., such as at about 60° C.

In some embodiments, the thermophilic host cell is a bacterium.

In further embodiments, the bacterium is selected from the group consisting of: Parageobacillus thermoglucosidasius, Geobacillus toebii, Geobacillus stearothermophilus, Geobacillus thermodenitrificans, Geobacillus kaustophilus, Geobacillus thermoleovorans, Geobacillus thermocatenulatus, Thermoanaerobacterium xylanolyticum, Thermoanaerobacterium saccharotyticum, Thermoanaerobacterium thermosaccharolyticum, Thermoanaerobacter mathranii, Thermoanaerobacter pseudoethanolicus, Thermoanaerobacter brockii, Thermoanaerobacter kivui, Thermoanaerobacter brockii, Caldanaerobacter subterraneus, Clostridium thermocellum, Clostridium thermosuccinogenes, Thermoclostridium stercorarium, Bacillus licheniformis, Bacillus coagulans, Bacillus smithii, Bacillus methanolicus, Bacillus flavothermus, Anoxybacillus kamchatkensis, Anoxybacillus gonensis, Caldicellulosiruptor bescii, Caldicellulosiruptor saccharolyticus, Caldicellulosiruptor kristjanssonii, Caldicellulosiruptor owensensis, Caldicellulosiruptor lactoaceticus, Moorella thermoacetica, Moorella thermoautotrophica, Thermus thermophilus, Thermus aquaticus, Thermotoga maritima, Pseudothermotoga lettingae, Pseudothermotoga thermarum, Chloroflexus aurantiacus, Anaerocellum thermophilum, Rhodothermus marinus, Sulfolobus acidocaldarius, Sulfolobus islandicus, Sulfolobus solfataricus, Thermococcus barophilus, Thermococcus kodakarensis, Pyrococcus abyssi, and Pyrococcus furiosus.

In a sixth aspect, the present invention relates to a thermophilic host cell comprising a heterologous expression control system comprising a DNA-binding protein, which is at least 80% identical to an amino acid sequence selected from: SEQ ID NOs: 1, 32-56, and 79-82, and a DNA sequence to which the DNA-binding protein can bind.

All embodiments of aspects 1-5 apply mutatis mutandis to the sixth aspect of the invention.

These and other aspects and embodiments of the invention are described in further detail below.

### LEGENDS TO THE FIGURES

FIG. 1: A) and B) mRuby2 expression over time at 52° C. for a wild type strain and a strain with the Tet_{R}-P_{tetR}-P_{tetA}-mRuby2 construct integrated, as determined by flow cytometry. Expression was induced by addition of anhydrotetracycline. AU=arbitrary units.

FIG. 2: A) β-galactosidase (BgaB) expression over time at 60° C. for a wild type strain and a strain with the Tet_{R}-P_{tetR}-P_{tetA}-BgaB construct integrated, as determined by absorbance measurements. Expression was induced by addition of anhydrotetracycline. The BgaB readout is expressed in Miller units. B) Growth curves of the cultures represented in (A), measured through OD₆₀₀.

FIG. 3: BgaB expression over time for a wild type strain and a strain with the Tet_{R}-P_{tetR}-P_{tetA}-BgaB construct integrated, as determined by absorbance measurements. Expression was induced by a shift in temperature. Both strains were measured in uninduced (60° C. constant) and induced (60° C. to 65° C. shift) states.

FIG. 4: Schematic illustration of the temperature regulation-principle of the present invention. At temperatures below 60° C., the tetR repressor represses the expression of the gene of interest (GOI), which is under the control of the tetA promoter. At temperatures above 65° C., the tetR protein is destabilized and can no longer repress expression from the tetA promoter, and thereby the GOI is expressed.

FIG. 5: Molecular dynamic (MD) simulation of the DNA-bound TetR homodimer, at low (dotted line) and high (solid line) temperature, shown as average root mean square fluctuation (RMSF) value in Ångström (Å) per residue of the crystal structure. Light grey shading indicates the conserved helix-turn-helix (HTH) region of the TetR family, where dark grey indicates the DNA-binding domain of the protein.

FIG. 6: Percentage identity matrix as a result of Multiple Alignment using Fast Fourier Transform (MAFFT) for all TetR family proteins used in this work. Shading indicates percentage identity score. SEQ ID NOs referring to the sequence listing of the amino acid sequences used for generating this data can be found in Table 9.

FIG. 7: Percentage identity matrix as a result of Multiple Alignment using Fast Fourier Transform (MAFFT) for the helix-turn-helix (HTH) regions of the TetR family proteins used in this work. Shading indicates percentage identity score. SEQ ID NOs referring to the sequence listing of the amino acid sequences used for generating this data can be found in Table 9.

FIG. 8: Difference in root mean square fluctuation (ΔRMSF in Ångström, Å) values for 8 representative proteins in the TetR family, when comparing low versus high temperature. The dotted line shows the average ΔRMSF values across the whole protein (x̅_{ΔRMSF}). Light grey shading indicates the helix-turn-helix (HTH) region, where dark grey depicts the DNA-binding domain of each protein. Destabilized regions are defined where ΔRMSF>1.5x̅_{ΔRMSE} for at least four consecutive residues, and their amino acid positions indicated in each plot. PDB ID numbers of crystal structures used for each protein can be found in Table 8.

### DETAILED DISCLOSURE OF THE INVENTION

### Definitions

As used herein, the term “thermophilic cell” refers to any cell, such as a microbial cell, e.g., a bacterial, eukaryotic or archaeal cell, which has an optimum growth temperature of at least about 40° C., such as at least about 45° C., such as at least about 50° C., such as at least about 55° C. Typically, a thermophilic cell as referred to herein has an optimum growth temperature in the range of about 40° C. to about 125° C. or about 45° C. to about 125° C. or about 55° C. to about 125° C., such as in the range of about 40° C. to about 80° C. or about 45° C. to about 80° C. or about 55° C. to about 80° C.

As used herein, the term “mesophilic cell” refers to any cell, such as a microbial cell, e.g., a bacterial cell, with has an optimum growth temperature of at most about 45° C., such as an optimum growth range in the range of about 20 to about 45° C. Non-limiting examples of mesophilic cells are bacteria of the Escherichia genera, such as Escherichia coli.

As used herein, a “host cell” is a cell, such as a thermophilic cell, which comprises at least one transgene, e.g., a heterologous gene.

As used herein, the term “Tet repressor” or “TetR” refers to a protein which is a member of the TetR family of transcriptional repressors found in bacterial cells, as well as functionally active fragments and variants thereof (see, e.g., Ramos et al., Microbiology and Molecular Biology Reviews 2005;69(2):326-356 and Cuthbertson and Nodwell, Microbiology and Molecular Biology Reviews 2013;77(3) doi:10.1128/MMBR.00018-13). At least some TetR proteins contains a helix-turn-helix (HTH) structural motif, which reportedly provides for their binding to the target DNA sequence (Ramos et al., 2005). Preferred TetR proteins are from mesophilic gram-negative bacterial cells. An example of a TetR protein is the Escherichia coli TetR protein (SEQ ID NO:1; UniProt ID: POACT4). A TetR protein according to the present invention may for example be a protein with at least about 80% sequence identity to SEQ ID NO: 1. Another example of a TetR protein is PfmR from the thermophilic bacterium Thermus thermophilus HB8 (SEQ ID NO: 74; UniProt ID: Q53WD9). A TetR protein typically binds to DNA in dimerized form, and, unless otherwise indicated or contradicted by context, the term “TetR protein” may refer to a protein in either monomeric or dimeric form.

As used herein, the term “tet operator” or “tetO” refers to a target DNA sequence of a TetR protein as described herein. The tetO is typically part of or associated with a promoter, whose ability to allow the expression of a gene to which the promoter is operably linked is regulated by the binding of the TetR protein to the tetO. Non-limiting tetO sequences include native tetO sequences from bacterial cells, such as mesophilic bacterial cells, as well as variants thereof where one or more mutations have been introduced. An example of a tetO sequence is the E. coli tetO1 sequence (SEQ ID NO:4).

As used herein, the term “gene” or “coding sequence” refers to a nucleic acid sequence that encodes a biological molecule, such as a protein, optionally including or operably linked to regulatory sequences preceding (5′ non-coding sequences) and following (3′ non-coding sequences) the coding sequence. A “transgene” is a gene that has been introduced into a cell, by a genetic engineering technique, such as by transformation. A transgene may be native or heterologous to the cell into which it is introduced. Gene names are herein set forth in italicised text with a lower-case first letter whereas protein names are set forth in normal text with a capital first letter.

Unless otherwise stated, “sequence identity”, as used for amino acid sequences or nucleotide sequences herein, is determined by comparing two optimally aligned sequences of equal length according to the following formula: (N_{ref}−N_{dif})·100/N_{ref}, wherein N_{ref} is the number of residues or nucleotides in one of the two sequences and Ndif is the number of residues or nucleotides which are non-identical in the two sequences when they are aligned over their entire lengths and in the same direction. Hence, the nucleotide sequence AGGTCCTA will have a sequence identity of 87.5% with the sequence AGTTCCTA (n_{dif}=1 and n_{ref}=8).

The sequence identity can be determined by conventional methods, e.g., Smith and Waterman (Adv. Appl. Math.; 2:482 1981), by the ‘search for similarity’ method of Pearson and Lipman (Proc. Natl. Acad. Sci. USA;85:2444 1988), using the CLUSTAL W algorithm of Thompson et al. (Nucleic Acids Res.; 22:467380 1994), by computerized implementations of these algorithms (GAP, BESTFIT, FASTA, and TFASTA in the Wisconsin Genetics Software Package, Genetics Computer Group), or the Needleman-Wunsch algorithm (Needleman and Wunsch, J. Mol. Biol.; 48: 443-453 1970) as implemented in the Needle program of the EMBOSS package (EMBOSS: The European Molecular Biology Open Software Suite, Rice et al., Trends Genet.; 16:276-277 2000), e.g., as provided at the European Bioinformatics Institute website (www.ebi.ac.uk). The BLAST algorithm (Altschul et al., Mol. Biol.; 215:403-410 1990), for which software may be obtained through the National Center for Biotechnology Information (www.ncbi.nlm.nih.gov/), may also be used. When using any of the mentioned algorithms, the default parameters for “Window” length, gap penalty, etc., may be used.

The term “expression”, as used herein, refers to the process in which a gene is transcribed into mRNA, and may optionally include the subsequent translation of the mRNA into an amino acid sequence, i.e., a protein or polypeptide.

As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell. An “active” promoter is a promoter from which a gene can be actively transcribed, e.g., because there are no transcription factors negatively regulating (i.e., repressing) gene expression from the promoter and/or because there are transcription factors present which positively regulate (i.e., promote) gene expression from the promoter. A “repressed” promoter is a promoter from which a gene cannot be actively transcribed or from which only a very low level of transcription occurs (so-called “leaky” expression). As used herein, “induced” expression means expression occurring from an active promoter, whereas when expression is “not induced”, it means that there is no expression or only leaky expression from a repressed promoter.

The term “operably linked,” when used in reference to a regulatory sequence and a coding sequence, means that the regulatory sequence can affect the expression of the linked coding sequence. “Regulatory sequences” or “control elements” refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences, etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule.

As used herein, the term “expression control system” refers to any system comprising at least two components and capable of controlling the expression of one or more genes of interest. Components of an expression control system may include, but are not limited to, one or more promoters, one or more genes to be expressed, one or more transcription factors and/or genes encoding said transcription factors, wherein the various components act together to control the expression of the one or more genes.

A “transcription factor” is a protein involved in the process of transcribing DNA into RNA. A transcription factor may, for example, initiate and/or regulate the transcription of genes, and usually comprises a DNA-binding domain that gives it the ability to bind to regulatory sequence such as, e.g., enhancer, promoter and operator sequences. One example of a transcription factor is a repressor, which is a DNA- or RNA-binding protein that inhibits the expression of one or more genes by binding to an operator sequence or associated silencer sequence.

The term “temperature regulation”, as used herein in the context of regulation of gene expression, refers to any means and mechanism of regulation that results in induction and/or abolishment of gene expression caused by a shift in temperature of the cell being regulated.

The term “optimum growth temperature”, as used herein about thermophilic cells, refers to a temperature or temperature interval where the growth-rate of the thermophilic cells is the highest.

By “tetracycline analog”, as used herein, is meant an analog of tetracycline (i.e., (4S,4aS,5aS,6S,12aR)-4-(dimethylamino)-1,6,10,11,12a-pentahydroxy-6-methyl-3,12-dioxo-4,4a,5,5a-tetrahydrotetracene-2-carboxamide; sold under the name of Sumycin among others), and capable of controlling a TetR-PtetA expression control system in a similar manner as tetracycline. A tetracycline analog preferably belongs to the tetracyclines family, containing the typical structure of at least four fused rings and triggering a conformational change in the TetR protein upon binding which causes it to lose its binding to its target DNA sequence. Examples of tetracycline analogs include, but are not limited to, anhydrotetracycline, doxycycline, demeclocycline, minocycline, chloro-tetracycline, sancycline, metacycline, and tigecycline (see, e.g., Krueger et al., Biotechniques;37 (4) doi: 10.2144/04374BM04 2018).

### Specific Embodiments of the Invention

As described in Example 1, it was found by the present inventors that a TetR-P_{tetA} expression control system retained its function, i.e. the expression of a gene under the control of the P_{tetA} promoter was repressed by TetR, when the system was used in the thermophilic bacterium Parageobacillus thermoglucosidasius at a temperature of 60° C. Surprisingly, when the temperature was raised to 65° C., expression of the reporter gene was induced.

Thus, the present inventors have unexpectedly discovered that expression control systems previously used in mesophilic host cells can be sufficiently stable also for use in thermophilic host cells at thermophilic temperatures. Furthermore, it was found that a TetR-P_{tetA} expression control system can, at thermophilic temperatures, be regulated by another mechanism than chemical induction by tetracycline, namely by temperature. Without being limited to theory, and as supported by the results in Example 2, a temperature increase may destabilize a TetR protein in a thermophilic cell, causing it to lose its ability to bind to the P_{tetA} promoter and thereby its ability to repress the expression of the gene whose expression it controls.

This discovery enables the use of this well-known expression control system also in thermophilic organisms, which are advantageous production hosts for fermentative bioproduction (as described in the background section). The toolbox available for controlling expression in these organisms is thereby expanded. Moreover, temperature-regulation of this expression control system enables its use without the external addition of inducer molecules, which can save expenses as well as decrease the likelihood of adding contaminants.

The expression control system can be used in any suitable thermophilic cell. Thermophilic cells may, for example, be isolated from natural sources (e.g., isolated from hot springs) or obtained from commercial sources. They may also potentially be generated in a laboratory, e.g., by adapting mesophilic cells to grow at higher temperatures by adaptive laboratory evolution. Preferably, in the context of the present invention, the thermophilic cell is a thermophilic bacterial cell, such as a gram-positive thermophilic bacterial cell.

Particularly contemplated are thermophilic host cells with an optimum growth temperature which is higher than about 40° C., such as at least about 45° C., such as at least about 50° C., such as at least about 55° C., such as at least about 56° C., at least about 57° C., such at least about 58° C., such as at least about 59° C., such as at least about 60° C. For example, the thermophilic host cell may have an optimum growth temperature in the range of about 40° C. to about 125° C., such as about 45° C. to about 125° C., such as about 50° C. to about 125° C., such as about 50° C. to about 100° C., such as about 50° C. to about 80° C., such as about 50° C. to about 75° C., such as about 50° C. to about 70° C., such as about 50° C. to about 65° C., such as about 50° C. to about 60° C., such as about 55° C. to about 125° C., such as about 55° C. to about 100° C., such as about 55° C. to about 80° C., such as about 55° C. to about 75° C., such as about 55° C. to about 70° C., such as about 60° C. to about 125° C., such as about 60° C. to about 100° C., such as about 60° C. to about 80° C., such as about 60° C. to about 75° C., such as about 60° C. to about 70° C., such as about 60° C. to about 65° C., such as about 50° C. to about 60° C., such as about 55° C. to about 65° C. For example, the thermophilic host cell may have an optimum growth temperature in the range of about 50° C. to about 65° C., such as about 55° C. to about 60° C., such as at about 60° C.

The optimum growth temperature of a thermophilic cell can be determined by methods known to a person of skill in the art. Typically, the growth rate of the thermophilic cells is measured over a range of about 20° C. to about 40° C. at temperature intervals of about 2° C. to 10° C. For the thermophilic cell in question, a growth medium is chosen which contains appropriate media components that facilitate growth. The effect of temperature on growth rate can then be determined from the exponential increase in optical density measured at 440 nm. If a more detailed assessment is needed, the assay can be repeated using e.g. different media components, media adjusted to a different pH, and/or a smaller temperature range which includes the initially determined optimum growth temperature but with a smaller interval between the growth temperatures, e.g., in the range of 1 to 5° C.

Non-limiting examples of thermophilic bacterial cells are those belonging to a genus selected from: Geobacillus, Parageobacillus, Thermoanaerobacterium, Thermoanaerobacter, Caldanaerobacter, Bacillus, Thermoclostridium, Anoxybacillus, Caldicellulosiruptor, Moorella, Thermus, Thermotoga, Pseudothermotoga, Chloroflexus, Anaerocellum, Rhodothermus, Sulfolobus, Thermococcus, Pyrococcus, and Clostridium.

For example, the host cell may be a bacterium belonging to a species selected from: Parageobacillus thermoglucosidasius, Geobacillus toebii, Geobacillus stearothermophilus, Geobacillus thermodenitrificans, Geobacillus kaustophilus, Geobacillus thermoleovorans, Geobacillus thermocatenulatus, Thermoanaerobacterium xylanolyticum, Thermoanaerobacterium saccharotyticum, Thermoanaerobacterium thermosaccharolyticum, Thermoanaerobacter mathranii, Thermoanaerobacter pseudoethanolicus, Thermoanaerobacter brockii, Thermoanaerobacter kivui, Thermoanaerobacter brockii, Caldanaerobacter subterraneus, Clostridium thermocellum, Clostridium thermosuccinogenes, Thermoclostridium stercorarium, Bacillus licheniformis, Bacillus coagulans, Bacillus smithii, Bacillus methanolicus, Bacillus flavothermus, Anoxybacillus kamchatkensis, Anoxybacillus gonensis, Caldicellulosiruptor bescii, Caldicellulosiruptor saccharolyticus, Caldicellulosiruptor kristjanssonii, Caldicellulosiruptor owensensis, Caldicellulosiruptor lactoaceticus, Moorella thermoacetica, Moorella thermoautotrophica, Thermus thermophilus, Thermus aquaticus, Thermotoga maritima, Pseudothermotoga lettingae, Pseudothermotoga thermarum, Chloroflexus aurantiacus, Anaerocellum thermophilum, Rhodothermus marinus, Sulfolobus acidocaldarius, Sulfolobus islandicus, Sulfolobus solfataricus, Thermococcus barophilus, Thermococcus kodakarensis, Pyrococcus abyssi, and Pyrococcus furiosus.

Non-limiting examples of thermophilic cells are bacteria of the Parageobacillus genus. Preferably, the thermophilic host cell is a Parageobacillus thermoglucosidasius. Other examples of thermophilic cells are Caldicellulosiruptor bescii, Clostridium thermocellum, and Moorella thermoacetica.

The expression control system introduced into the thermophilic cell may comprise a TetR protein and a first promoter comprising a tetO sequence to which the TetR protein can bind. The expression control system or the thermophilic host cell may also further comprise a gene encoding the TetR protein, optionally wherein the gene encoding the TetR protein is operatively linked to a second promoter. Alternatively, the expression control system introduced into the thermophilic cell may comprise a gene encoding a TetR protein, a first promoter comprising a tetO sequence to which the TetR protein can bind, wherein the gene encoding the TetR protein is operatively linked to a second promoter. Typically, in the expression control system or the thermophilic host cell, the first promoter is operatively linked to a gene of interest. The expression control system may further comprise one or more additional regulatory sequences as described elsewhere herein, e.g., additional tetO sequences.

In one non-limiting embodiment, the expression control system introduced into the thermophilic host cell comprises the following components, e.g., as a part of one or more nucleic acid constructs, such as a plasmid:
(i) a P_{tetA} promoter comprising a first tetO (tetO1) sequence to which the TetR protein can bind and operatively linked to a gene of interest;
(ii) a gene encoding a TetR protein, operatively linked to a P_{tetR} promoter which comprises a second tetO (tetO2) sequence to which the TetR protein can bind;
(iii) An origin of replication, suited for Gram-negative, mesophilic hosts. E.g., BBR1;
(iv) An origin of replication suited for the thermophilic host cell, e.g., RepB, and/or two homology arms, allowing genome integration of the construct;
(v) A selection marker suitable for selection in mesophiles and/or thermophiles. E.g., a kanamycin resistance gene;
(vi) Terminators.

Although it is contemplated that one or more components of the expression control system may exist naturally in (i.e., be endogenous to) the thermophilic cell, at least one component of the expression control system is typically heterologous to the thermophilic cell.

In some cases, the thermophilic host cell may take up a synthesized linear DNA strand, either single-or double-stranded, in the absence of a plasmid vector. Thus, in some embodiments, a linear piece of DNA encoding the expression control system according to the invention and sequences for homologous recombination may be taken up by the cell and become integrated into its genome. The host cell may alternatively by transduced with a viral vector encoding the expression control system.

As described herein, the temperature-regulated expression control system can provide for thermophilic host cells, uses and methods characterized by the following: When the thermophilic host cell is incubated in a culture medium having a first temperature, the expression of the gene of interest is not induced, whereas when the thermophilic host cell is incubated in a culture medium having a second temperature, the expression of the gene of interest is induced. The second temperature may be at least about 3° C., such as at least about 4° C., such as at least about 5° C., such as at least about 6° C., such as at least about 7° C., such as at least about 8° C., such as at least about 9° C., such as at least about 10° C. higher than the first temperature, e.g., at least 15° C., 20° C., or 25° C. higher, such as at most 30° C. higher than the first temperature. The second temperature may, for example, be at least about 4° C. higher than the first temperature. The second temperature may alternatively be at least about 5° C. higher, such as about 5° C. higher, than the first temperature.

The first and second temperatures can be selected depending on the characteristics of the expression control system in question as well as on the growth characteristics of the thermophilic cell. It may be advantageous if the expression control system provides for temperature-based regulation at temperatures close to the optimum growth temperature of the thermophilic host cell in question. Accordingly, the first temperature, the second temperature, or both may be within about 5° C., such as within about 10° C., of the optimum growth temperature. However, in other cases it may be more beneficial if the expression control system provides regulation at temperatures distant from the optimum growth temperature, since this may provide for separate phases of growth and production.

Many TetR proteins are known in the art (see, e.g., Ramos et al., 2005). TetR proteins suitable for the expression control systems, thermophilic host cells, uses and methods described herein are not limited to any specific TetR proteins, originating from or expressed in any particular species. Particularly contemplated are, however, TetR proteins from mesophilic bacteria and functionally active fragments and variants thereof. Non-limiting examples of TetR proteins include those shown in Table 1, those shown in Table 8, as well as functionally active variants and fragments thereof. Particularly preferred is a TetR protein comprising the amino acid sequence of SEQ ID NO:1, or a functionally active fragment or variant thereof. In some embodiments, the TetR protein has the sequence of SEQ ID NO:1. Functionally active fragments and variants of, e.g., E. coli TetR (SEQ ID NO:1; 207 amino acids) can be identified by a person of skill in the art.

TABLE 1
Examples of TetR proteins

| | % identity to | |
|---|---|---|
| | E. coli TetR | Database Reference |
| Organism | (SEQ ID NO: 1) | Sequence |
| Escherichia coli | 100 | P0ACT4 (UniProt ID) |
| | | (SEQ ID NO: 1) |
| Providencia stuartii | 99.52 | WP_183124523.1 (NCBI) |
| | | (SEQ ID NO: 79) |
| Enterobacter | 99.52 | WP_165473055.1 (NCBI) |
| roggenkampii | | (SEQ ID NO: 80) |
| Shigella flexneri | 99.50 | WP_072195351.1 (NCBI) |
| | | (SEQ ID NO: 81) |
| Acinetobacter baumannii | 99.33 | WP_047939682.1 (NCBI) |
| | | (SEQ ID NO: 82) |

For example, the full-length sequence can be truncated from the N- and/or C-terminus, typically to remove residues or portions not necessary for its DNA-binding activity and/or expression control activity. For example, such a fragment may comprise at least about 60%, such as at least about 70%, such as at least about 80%, such as at least about 90%, such as at least about 95%, of the full length of the protein, counting by its amino acid residues in the native, mature sequence. Alternatively, such a fragment can be obtained by removing, e.g., 1, 2, 3, 4, 5, 10, 20 or more amino acid residues from the C-terminus, N-terminus, or both. In TetR proteins where the HTH motif is located in the N-terminal portion, the fragment is preferably obtained by removing one or more amino acids only from the C-terminal portion.

Variants comprising one or more amino acid substitutions, deletions or insertions can also be prepared and identified by screening or testing them for their DNA-binding activity and/or expression control activity. For example, a TetR protein as described herein may comprise an amino acid sequence at least about 60%, such as at least about 65%, such as at least about 70%, such as at least about 75%, such as at least about 80%, such as at least about 85%, such as at least about 90%, such as at least about 95%, such as at least about 96%, such as at least about 97%, such as at least about 98%, such as about 99%, identical to any one of the sequences with SEQ ID NOs: 1, 32-56, and 79-82, more preferably to that of E. coli TetR (SEQ ID NO: 1). Alternatively, such a variant may comprise up to 10 mutations, such as 1, 2, 3, 4, 5, 6, 7, 8, 9 or 10 mutations. In some particularly contemplated TetR variants, the variant differs from the native sequence only by conservative amino acid substitutions.

Variants of E. coli TetR are known in the art. For example, Ettner et al. (1996) generated 50 single amino acid TetR variants. Muller et al. (1995) generated tetR variants which did not respond to tetracycline inducer but most retained their DNA binding. The mutations were introduced as single amino acid substitutions distributed in different secondary structures throughout the protein sequence; predominantly in the interior of the protein. Only 6 of the 93 variants tested showed decreased binding to tetO. Kamionka et al. (2004) generated a reverse tetR (i.e. which binds to tetO in the presence of tetracycline but does not bind to tetO in the absence of tetracycline) by introducing 2 point mutations; G96E and L205S. Scholz et al. (2004) introduced mutations to change TetR from responding to tetracycline to another inducer; 4-de(dimethylamino)-6-deoxy-6-demethyl-tetracycline (cmt3). The variants still bound to tetO in absence of inducer. Additionally, since the crystal structure of E. coli TetR in complex with tetO has been reported (Orth et al., 2001), amino acids suitable for substitution or deletion can be identified and tested. Generally, protein segments outside of the secondary structures (α-helices and β-strands) are more flexible and would allow for mutagenesis without causing changes in functionality. Moreover, conservative amino acid substitutions (e.g., R to K mutations or E to D mutations) can be introduced.

A “conservative” amino acid substitution in a protein is one that does not negatively influence protein activity. Typically, a conservative substitution can be made within groups of amino acids sharing physicochemical properties, such as, e.g., basic amino acids (arginine, lysine and histidine), acidic amino acids (glutamic acid and aspartic acid), polar amino acids (glutamine and asparagines), hydrophobic amino acids (leucine, isoleucine, valine and methionine), aromatic amino acids (phenylalanine, tryptophan and tyrosine), and small amino acids (glycine, alanine, serine, and threonine). Most commonly, substitutions can be made between Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, Asp/Gly.

In some embodiments, the C- or N-terminus of the TetR protein, or functionally active variant or fragment thereof, is fused to a second polypeptide, such as a protein tag or linker. Suitable protein tags for facilitating detection of a protein or for improving stability, solubility or other properties of a protein are well-known in the art. Non-limiting proteins tags for facilitating protein detection include Green Fluorescent protein (GFP) and Red Fluorescent Protein (RFP). Linkers which may, for example, be used as spacers in fusion proteins of a TetR protein with another protein include (G₄S)₄-His₆.

Suitable assays for testing the expression control activity (and, without being limited to theory, thereby also the DNA binding activity) of such fragments or variants or fusion proteins in an expression control system are provided in Example 1. For example, a P. thermoglucosidasiusDSM2542Z Δldh::TetR-PtetR-PtetA-BgaB test strain can be prepared where the gene encoding the E. coli TetR protein is replaced by a gene encoding the fragment or variant to be tested. Preferably, when the thermophilic host cell is incubated in a culture medium having a first temperature, the expression of the gene of interest is not induced; when the thermophilic host cell is incubated in a culture medium having a second temperature, the expression of the gene of interest is induced; and the second temperature is at least about 4° C. higher than the first temperature, e.g., at least about 5° C. higher than the first temperature. For example, the first temperature may be about 60° C. and the second temperature about 65° C.

As described in Example 1, the coding sequence for a TetR protein may be modified to remove predicted restriction sites and for the purpose of codon optimization. For example, the coding sequence of SEQ ID NO: 2 may be modified to SEQ ID NO: 3 (see Table 4).

In some embodiments, the TetR protein may have a melting temperature between 40° C. and 90° C., such as between 45° C. and 55° C., between 50° C. and 60° C., between 55° C. and 65° C., between 60° C. and 70° C., between 65° C. and 75° C., between 70° C. and 80° C., between 75° C. and 85° C., or between 80° C. and 90° C.

In some embodiments, the TetR protein comprises a helix-turn-helix (HTH) region, which comprises a DNA-binding domain and a sub-region of at least 4 consecutive amino acid residues, which sub-region destabilizes upon temperature increase.

In further embodiments, the sub-region has a change in root mean square fluctuations (ΔRMSF) score above 1.5 times the average ΔRMSF score for the whole protein in dimeric form when measured by molecular dynamic analysis using the software program CABS-flex 2.0.

The calculation of ΔRMSF scores is described in Example 2.

The sub-region may comprise between 4 and 100 consecutive amino acid residues, such as at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, and such as at least 50, consecutive amino acid residues.

The sub-region may overlap with the DNA-binding domain either partially or fully, or it may not overlap with the DNA-binding domain at all.

In some embodiments, the sub-region overlaps with the DNA-binding domain.

In some embodiments, the sub-region overlaps fully with the DNA-binding domain.

In some embodiments, the sub-region overlaps partially with the DNA-binding domain.

In some embodiments, the sub-region does not overlap with the DNA-binding domain.

By “overlaps fully” is meant that the whole sub-region is comprised within the boundaries of the DNA-binding domain. By “overlaps partially” is meant that only a part of the sub-region, such as at least 1, at least 2, at least 3, or at least 4 consecutive amino acid residues of the sub-region, is within the boundaries of the DNA-binding domain.

In some embodiments, a TetR protein may comprise a sub-region comprising or consisting of:
(a) amino acid residues 26-47 of tetR (SEQ ID NO:1);
(b) amino acid residues 36-62 of amtR (SEQ ID NO:33);
(c) amino acid residues 31-42 of icaR (SEQ ID NO:37);
(d) amino acid residues 26-44 of ImrA (SEQ ID NO:32);
(e) amino acid residues 36-46 of qacR (SEQ ID NO:36);
(f) amino acid residues 39-58 of smcR (SEQ ID NO:41);
(g) amino acid residues 18-46 of pfmR (SEQ ID NO:51);
(h) amino acid residues 23-53 of fadR (SEQ ID NO:53); or
(i) a variant of any one of (a) to (h), which has 1, 2 or 3 amino acid substitutions, deletions or insertions.

Preferably, the sub-region domain overlaps at least partially with the DNA binding domain.

The expression control system introduced into the thermophilic host cell may comprise a second promoter which is operatively linked to a gene encoding the TetR protein. Suitable second promoters which can induce the expression of a TetR protein to which it is linked in a thermophilic bacterial cell can be chosen by a person of skill in the art depending on, e.g., the thermophilic host cell and/or the TetR protein. In theory any constitutive promoter could be used, as long as it works in the organism of choice at a high enough level to have sufficient TetR protein to allow for controlling the promoter(s) containing the tetO binding sequence. It could also be in a circuit (e.g., be controlled by another input) as well as be designed to be self-regulated. As an example, the gene encoding the TetR protein may be operatively linked to the PXyIR promoter. As described in Example 1, the second promoter may be a modified PXyIR promoter, such as the one with SEQ ID NO: 13, which is originally derived from Bacillus subtilis. This promoter is based on the pRMC2 plasmid from Corrigan et al., 2009, wherefrom the modified B. subtilis PXyIR promoter was copied. The −10 box was then replaced with the B. subtilis consensus sequence (Corrigan et al., 2009; Fagan and Fairweather, 2011). Suitable promoters also include any native promoter which regulates the expression of a native TetR protein in a bacterial cell, e.g., an E. coli cell. Preferred promoters are at least about 85%, such as at least about 90% or 100% identical to the promoter with SEQ ID NO: 13.

The expression control system may comprise a first promoter which comprises a tetO sequence which can be bound by the TetR protein, e.g., the E. coli TetR protein having the amino acid sequence of SEQ ID NO: 1, and/or fragments or variants thereof. Many tetO sequences are known in the art. The tetO sequence suitable for the expression control systems, thermophilic host cells, uses and methods described herein are thereby not limited to any specific tetO sequence, originating from or present in any particular species. Moreover, variants of native tetO sequences can be prepared and tested for their binding activity to a TetR protein as described herein. Preferred tetO sequences include those that comprise a nucleotide sequence having at least about 65%, such as at least about 70%, such as at least about 75%, such as at least about 80%, such as at least about 85%, such as at least about 90%, and such as at least about 95%, such as 100% sequence identity to the tetO1 sequence tetO1-pRMC2 (SEQ ID NO: 5). For example, it has previously been shown that certain mutated tetO sequences having a sequence identity to the tetO1 sequence from E. coli (SEQ ID NO:4, 94.7% identical to SEQ ID NO:5) of 68.4% still retain the ability to be bound by TetR proteins (Reichheld et al., 2009, Sizemore et al., 1990). Some specifically contemplated tetO sequences and variants thereof are shown in Table 2. Preferably, the tetO sequence comprises SEQ ID NO: 5.

TABLE 2
Examples of tetO sequences

| | | | SEQ |
|---|---|---|---|
| | | % identity | ID |
| Name | 5′→3′ Sequence | to tetO1 | NO |
| tetO1 ¹ | ACTCTATCAATGATAGAGT | 100 | 4 |
| tetO1-pRMC2 ² | ACTCTATCATTGATAGAGT | 94.7 | 5 |
| tetO2 ³ | TCCCTATCAGTGATAGAGA | 78.9 | 6 |
| tetO2-M3 ⁴ | TCCCTAACAGTGTTAGAGA | 68.4 | 7 |
| tetO2-M4 ⁴ | TCCCTTTCAGTGAAAGAGA | 68.4 | 8 |
| tetO2-M5 ⁴ | TCCCAATCAGTGATTGAGA | 68.4 | 9 |
| tetO2-M6 ⁴ | TCCGTATCAGTGATACAGA | 68.4 | 10 |
| tetO2-pWH1012- | TCCCCATCAGTGATGGAGA | 68.4 | 11 |
| 5T ⁵ | | | |

¹ Fagan et al., 2011; ² Corrigan et al., 2009; ³ Bertram et al., 2007; ⁴ Reicheld et al., 2009; ⁵ Sizemore et al., 1990

Any promoter or promoter region which can include a tetO sequence to which the TetR protein can bind and which can be operatively linked to at least one gene of interest can be used as the first promoter in the expression control systems, thermophilic host cells, uses and methods described herein. Suitable first promoters which can induce the expression of a gene of interest to which it is linked in a thermophilic bacterial cell can be chosen by a person of skill in the art depending on, e.g., the thermophilic host cell and/or the gene of interest. Preferably, a first promoter should have a suitable −10 and −35 site and a suitable tetO binding site. These sites could possibly be adjusted to give the system a preferred induction temperature and/or a preferred level of leakiness. Furthermore, the sequence prior to the gene of interest should preferably have an appropriate ribosomal binding site. Suitable promoters also include any native promoter which regulates the expression of a native TetA protein in a bacterial cell, e.g., an E. coli cell, as well as any modified promoter derived from such native promoter. As described in Example 1, a suitable promoter may be a modified P_{tetA} promoter originally derived from E. coli, such as the promoter with SEQ ID NO: 12. This promoter is based on the pRPF185 plasmid from Fagan and Fairweather, 2011, wherefrom the modified E. coli Tn10 P_{tetA} sequence, adapted for use in Gram positive bacteria, was copied. As is also described in Example 1, the promoter with SEQ ID NO: 12 may be further optimized by removing possible restriction sites, resulting in the promoter with SEQ ID NO:14. Preferred promoters are at least about 85%, such as at least about 90% or 100% identical to the promoter with SEQ ID NO: 12 or at least about 80%, such as at least about 85%, such as at least about 90% or 100% identical to the promoter with SEQ ID NO: 14.

In particular thermophilic host cells, uses and methods, the TetR protein comprises an amino acid sequence at least 60% identical to SEQ ID NO: 1 and/or the tetO sequence comprises a nucleotide sequence at least 65% identical to SEQ ID NO: 5. For example, in the thermophilic host cell, use or the method, the expression control system may comprise a TetR protein comprising an amino acid sequence at least 90% identical to SEQ ID NO: 1, a tetO nucleotide sequence at least 90% identical to SEQ ID NO:5, a first promoter comprising a nucleotide sequence at least 90% identical to SEQ ID NO:14, and, optionally, a second promoter comprising a nucleic acid sequence at least 90% identical to SEQ ID NO: 13.

Constructs, e.g., a vector such as a plasmid, comprising (or encoding for) the components of the expression control system described herein can be introduced into the thermophilic host cell using a standard protocol, e.g., a protocol similar to the one used in Example 1. Any suitable procedure for introducing heterologous polynucleotide sequences into thermophilic host cells can be used. For example, only a single copy of each component of the expression control system and transgene may be introduced. Alternatively, more than a single copy, such as two copies, three copies or more than three copies of the expression control system components and transgene may be introduced. As is understood by the skilled artisan, multiple copies of heterologous polynucleotides may be on a single construct or on more than one construct.

Contemplated are thermophilic host cells where the components of the expression control system (or genes encoding them) are transiently introduced into the thermophilic host cell through use of a plasmid or shuttle vector. Preferably, however, the components of the expression control system (or genes encoding them) are permanently introduced into a chromosome of the thermophilic host cell. Chromosomal integration techniques are known to the skilled artisan and have been described in, for example, Zhou and Wolk, J Bacterid., 184 (9): 2529-2532, 2002. Briefly, polynucleotide sequences comprising (or encoding) the components of the expression control system and gene of interest may be subcloned into one or more integration vectors. This construct may then be introduced into the thermophilic host cell for double homologous recombination at specific loci on the host cell chromosome, e.g., the lactate dehydrogenase (Idh) locus.

In the thermophilic host cell as described herein, the first promoter is operatively linked to a gene of interest. A wide variety of genes/gene products may be relevant to express/produce from the gene of interest according to the uses and methods described herein. The gene product could for example be a protein which is already produced industrially in a thermophilic microorganism but using a different expression control system than the Tet_{R}-P_{tetA} system or using a Tet_{R}-P_{tetA} system but with tetracycline induction. The gene product could also be a protein endogenously produced in a mesophilic bacterium, but which could more advantageously be produced in a thermophilic organism using the expression control system according to the present invention. The gene product could also be an RNA sequence transcribed from the gene of interest. Table 3 gives some examples of suitable genes and gene products for expression/production in thermophilic host cells. Typically, the gene product of interest is not a TetR protein.

TABLE 3
Examples of suitable genes/gene products of interest

| Gene name | Protein name | Comments/references |
|---|---|---|
| alsS and alsD | Acetolactate synthase | Can be from various sources, e.g. B. |
| | and acetolactate | subtilis, for the production of 2,3- |
| | decarboxylase, | butanediol or acetoin. (Zhou et al., Appl. |
| | respectively. | Microbiol. Biotechnol. 2020) |
| ribC | Riboflavin synthase | This gene can be used to get the |
| | | thermophilic cell to produce riboflavin. |
| | | (Yang et al., Microb. Biotechnol. 2020) |
| kivD, ilvC, alsS, | 2-ketoisovalerate | These genes form a pathway that can be |
| ilvD | decarboxylase, ketol- | used for the production of isobutanol in |
| | acid reductoisomerase, | thermophiles. (Lin et al., Metabol. |
| | acetolactate synthase, | Engineer. 2014) |
| | dihydroxy-acid | |
| | dehydratase | |
| roseRS_3509, | Terpene synthase, | These genes form a pathway for the |
| hmgr, aca, hmgs, | hydroxymethylglutaryl- | production of terpenes. (Styles et al., |
| mvk, dmd, pmk | CoA reductase, acetyl- | Metabol. Engineer. 2021) |
| and idi, fpps | CoA acetyltransferase, | |
| | hydroxymethylglutaryl- | |
| | CoA synthase, | |
| | mevalonate kinase, | |
| | diphosphomevalonate | |
| | decarboxylase, | |
| | isopentenyl | |
| | pyrophosphate | |
| | isomerase, FPP synthase | |
| xynA1 | Xylanase | A xylanase from a Parageobacillus strain |
| | | was produced in a different |
| | | Parageobacillus strain, for production of |
| | | enzymes required for biomass |
| | | degradation. (Holland et al., BMC |
| | | Biotechnol. 2019) |

Provided are also methods and uses of the expression control system and thermophilic host cells as described herein. In some expression systems, when the thermophilic host cell is incubated in a culture medium having a first temperature, the expression of the gene of interest is not induced; when the thermophilic host cell is incubated in a culture medium having a second temperature, the expression of the gene of interest is induced; and the second temperature is at least about 4° C. higher than the first temperature. In some expression systems, methods and uses described herein, the temperature is increased from a temperature of up to 60° C. to a temperature of at least 65° C. For example, the first temperature may be ≤59° C., such as 55° C., such as 50° C., and such as 45° C., and the second temperature may be ≥66° C., such as 70° C., such as 75° C., and such as 80° C. In preferred embodiments, the first temperature is around 60° C. and the second temperature is around 65° C.

In some expression systems described herein, an increase in temperature results in induction of expression of the gene of interest. Accordingly, these expression systems are useful for uses and methods where, at the first (lower) temperature, the thermophilic host cell culture is multiplied, i.e., grown, and, once a desired number or concentration of thermophilic host cells has been achieved, the temperature is increased and the transcription and expression of the gene or protein of interest induced. The resulting biological molecule can then be isolated, e.g., retrieved from the cell culture and purified as needed.

TABLE 4
Sequences of specific expression control system components

| Sequence name | Sequence | SEQ ID NO |
|---|---|---|
| E. coli TetR amino acid | MSRLDKSKVINSALELLNEVGIEGLTTRKLAQ | 1 |
| sequence | KLGVEQPTLYWHVKNKRALLDALAIEMLDRH | |
| | HTHFCPLEGESWQDFLRNNAKSFRCALLSHR | |
| | DGAKVHLGTRPTEKQYETLENQLAFLCQQGF | |
| | SLENALYALSAVGHFTLGCVLEDQEHQVAKE | |
| | ERETPTTDSMPPLLRQAIELFDHQGAEPAFLF | |
| | GLELIICGLEKQLKCESGS | |
| E. coli TetR nucleotide | ATGATGTCTAGATTAGATAAAAGTAAAGTGA | 2 |
| sequence | TTAACAGCGCATTAGAGCTGCTTAATGAGG | |
| | TCGGAATCGAAGGTTTAACAACCCGTAAAC | |
| | TCGCCCAGAAGCTAGGTGTAGAGCAGCCTA | |
| | CATTGTATTGGCATGTAAAAAATAAGCGGG | |
| | CTTTGCTCGACGCCTTAGCCATTGAGATGTT | |
| | AGATAGGCACCATACTCACTTTTGCCCTTTA | |
| | GAAGGGGAAAGCTGGCAAGATTTTTTACGT | |
| | AATAACGCTAAAAGTTTTAGATGTGCTTTAC | |
| | TAAGTCATCGCGATGGAGCAAAAGTACATT | |
| | TAGGTACACGGCCTACAGAAAAACAGTATG | |
| | AAACTCTCGAAAATCAATTAGCCTTTTTATG | |
| | CCAACAAGGTTTTTCACTAGAGAATGCATTA | |
| | TATGCACTCAGCGCTGTGGGGCATTTTACT | |
| | TTAGGTTGCGTATTGGAAGATCAAGAGCAT | |
| | CAAGTCGCTAAAGAAGAAAGGGAAACACCT | |
| | ACTACTGATAGTATGCCGCCATTATTACGAC | |
| | AAGCTATCGAATTATTTGATCACCAAGGTGC | |
| | AGAGCCAGCCTTCTTATTCGGCCTTGAATT | |
| | GATCATATGCGGATTAGAAAAACAACTTAAA | |
| | TGTGAAAGTGGGTCTTAA | |
| Modified E. coli TetR | ATGATGTCTAGATTAGATAAAAGTAAAGTGA | 3 |
| nucleotide sequence (used in | TTAACAGCGCATTAGAGCTGCTTAATGAGG | |
| Ex. 1) | TCGGAATCGAAGGTTTAACAACCCGTAAAC | |
| | TCGCCCAGAAGCTAGGTGTAGAGCAGCCTA | |
| | CATTGTATTGGCATGTAAAAAATAAGCGGG | |
| | CTTTGCTCGACGCCTTAGCCATTGAGATGTT | |
| | AGATAGGCACCATACTCACTTTTGCCCATTA | |
| | GAAGGGGAAAGCTGGCAAGATTTTTTGAGA | |
| | AATAACGCTAAAAGTTTTAGATGTGCTTTAC | |
| | TAAGTCATCGCGATGGGGCAAAAGTACATT | |
| | TAGGTACACGGCCTACAGAAAAACAGTATG | |
| | AAACTCTCGAAAATCAATTAGCCTTTTTATG | |
| | CCAACAAGGTTTTTCACTAGAGAATGCATTA | |
| | TATGCACTCAGCGCTGTTGGTCATTTTACTT | |
| | TAGGTTGCGTATTGGAAGATCAAGAGCATC | |
| | AAGTCGCTAAAGAAGAAAGGGAAACACCTA | |
| | CTACTGATAGTATGCCGCCATTATTACGACA | |
| | AGCTATCGAATTATTTGATCATCAAGGGGC | |
| | AGAGCCAGCCTTCTTATTCGGCCTTGAATT | |
| | GATCATATGCGGATTAGAAAAACAACTTAAA | |
| | TGTGAAAGTGGGTCTTAA | |
| E. coli tetO1 sequence | ACTCTATCAATGATAGAGT | 4 |
| tetO1-pRMC2 (used in Ex. 1) | ACTCTATCATTGATAGAGT | 5 |
| tetO2 | TCCCTATCAGTGATAGAGA | 6 |
| tetO2-M3 | TCCCTAACAGTGTTAGAGA | 7 |
| tetO2-M4 | TCCCTTTCAGTGAAAGAGA | 8 |
| tetO2-M5 | TCCCAATCAGTGATTGAGA | 9 |
| tetO2-M6 | TCCGTATCAGTGATACAGA | 10 |
| tetO2-pWH1012-5T | TCCCCATCAGTGATGGAGA | 11 |
| P_{tetA} sequence (modified, | TTGACACTCTATCATTGATAGAGTATAATTA | 12 |
| originally from E. coli ¹) | AAATAAGCTTGATCGTAGCGTTAACAGATCT | |
| | GAGCTCCTGCAGTAAGCTG | |
| P_{tetR} sequence (based on a | TGACAAATAACTCTATCAATGATATAATGTC | 13 |
| modified B. subtilis PXyIR | AACAAAAAGGAGGAATTA | |
| promoter ^{1,2} - used in Ex. 1) | | |
| P_{tetA} sequence (modified, | TTGACACTCTATCATTGATAGAGTATAATTA | 14 |
| originally from E. coli ¹ - used | AAATAAGCTTGATCGTAGCGATTACAGATCT | |
| in Ex. 1) | GTCCTCCTGCAGTAAGCTG | |
| mRuby2 amino acid sequence | MVSKGEELIKENMRMKVVMEGSVNGHQFKC | 15 |
| (used in Ex. 1) | TGEGEGNPYMGTQTMRIKVIEGGPLPFAFDIL | |
| | ATSFMYGSRTFIKYPKGIPDFFKQSFPEGFTW | |
| | ERVTRYEDGGVVTVMQDTSLEDGCLVYHVQ | |
| | VRGVNFPSNGPVMQKKTKGWEPNTEMMYPA | |
| | DGGLRGYTHMALKVDGGGHLSCSFVTT | |
| BgaB amino acid sequence | MNVLSSICYGGDYNPEQWPEEIWYEDAKLM | 16 |
| (used in Ex. 1) | QKAGVNLVSLGIFSWSKIEPSDGVFDFEWLD | |
| | KVIDILYDHGVYINLGTATATTPAWFVKKYPD | |
| | SLPIDESGVIFSFGSRQHYCPNHPQLITHIKR | |
| | LVRAIAERYKNHPALKMWHVNNEYACHVSK | |
| | CFCENCAVAFRKWLKERYKTIDELNERWGTN | |
| | FWGQRYNHWDEINPPRKAPTFINPSQELDYY | |
| | RFMNDSILKLFLTEKEILRGVTPDIPVSTNFM | |
| | GSFKPLNYFQWAQHIDIVTWDSYPDPREGLP | |
| | IQHAMMNDLMRSLRKGQPFILMEQVTSHVN | |
| | WRDINVPKPPGVMRLWSYATIARGADGIMFF | |
| | QWRQSRAGAEKFHGAMVPHFLNENNRIYRE | |
| | VTQLGQELKKLDCLVGSRIKAEVAIIFDWEN | |
| | WWAVELSSKPHNKLRYIPIVEAYYRELYKRNI | |
| | AVDFVRPSDDLTKYKVVIAPMLYMVKEGEDE | |
| | NLRQFVANGGTLIVSFFSGIVDENDRVHLGG | |
| | YPGPLRDILGIFVEEFVPYPETKVNKIYSNDGE | |
| | YDCTTWADIIRLEGAEPLATFKGDWYAGLPA | |
| | VTRNCYGKGEGIYVGTYPDSNYLGRLLEQVF | |
| | AKHHINPILEVAENVEVQQRETDEWKYLIIIN | |
| | HNDYEVTLSLPEDKIYQNMIDGKCFRGGELRI | |
| | QGVDVAVLREHDEAGKV | |
| Additional TetR proteins | | SEQ ID |
| | | NOs: 32-56 |
| | | and 79-82 |

¹ Fagan et al., 2011; ² Corrigan et al., 2009

### EXAMPLE 1

### Construction of Inducible Expression System

This example demonstrates the construction of an inducible promoter system, where a shift in temperatures within the thermophilic range is used to activate expression from the promoter system. The functionality of the system is shown with a thermostable β-galactosidase.

For stable and homogeneous expression of constructs, integrative expression is preferred over plasmid-based expression. Previous work showed the suitability of the lactate dehydrogenase (Idh) locus (GenBank: ALF09597.1) for the integration of target constructs (Sheng et al., Biotechnol. Biofuels; 10:1-18 2017). First, a plasmid backbone that allows the integration of target constructs into the Idh locus was created. Homologous regions of 1000 base pairs, flanking the genomic region that was to be modified, were amplified using polymerase chain reaction (PCR). The primers used are shown in Table 5—primer sequences are shown in Table 7. The flanks were inserted into a pMTL61110 (Sheng et al., Biotechnol. Biofuels; 10:1-18 2017) backbone through standard USER cloning (Bitinaite et al., Nucleic Acids Res.; 35:1992-20022007) approaches, to create pMTL61110-Δldh. To insert the final temperature sensitive inducible system into the integrative backbone vector, an initial iteration using the endogenous nucleotide sequence of the gene encoding the Escherichia coli TetR protein (SEQ ID NO:2 (gene) and SEQ ID NO:3 (protein)) and the P_{tetR}-P_{tetA} promoter sequence (SEQ ID NO:13 (PtetR) and SEQ ID NO: 12 (PtetA)) from the plasmid pRPF185 (Fagan and Fairweather, 2011) was performed. As described above, the P_{tetR} promoter sequence is a modified PXyIR promoter originally derived from B. subtilis. It was originally copied from the pRMC2 plasmid (Corrigan et al., 2009), and the −10 box was replaced with the B. subtilis consensus sequence (Fagan and Fairweather, 2011). The P_{tetA} promoter sequence is originally derived from the E. coli Tn10 P_{tetA} but has been adapted for use in Gram positive bacteria (Fagan and Fairweather, 2011). The Tet_{R}-P_{tetR}-P_{tetA} region was amplified from pRPF185 by PCR, with the primer pair as indicated in Table 5. To evaluate the performance of the expression system, two different reporter proteins were used. To evaluate functioning at 52° C., mRuby2 (SEQ ID NO: 15) was used as a fluorescent marker. The marker was amplified from pcDNA3-mRuby2 using primers indicated in Table 5.

As mRuby2 does not function above 53° C., to evaluate the systems functioning above 53° C., a thermostable β-galactosidase from Geobacillus stearothermophilus (BgaB, SEQ ID NO: 16) was selected as reporter protein and PCR amplified from pMTLP13B using primers indicated in Table 5 (Ølshøj Jensen et al., AMB Express;7 2017). The PCR products were cloned into pMTL61110-Δldh using USER cloning, resulting in pMTL61110-Δldh-Tet_{R}-P_{tetR}-P_{tetA}-mRuby2 and pMTL61110-Δldh-Tet_{R}-P_{tetR}-P_{tetA}-BgaB. The resulting construct was verified by Sanger sequencing.

To test the construct in the model thermophile Parageobacillus thermoglucosidasius DSM2542^{z}, transformation of the pMTL61110-Δldh-Tet_{R}-P_{tetR}-P_{tetA}-mRuby2 and pMTL61110-Δldh-Tet_{R}-P_{tetR}-_{tetA}-BgaB plasmids was performed through a high osmolarity electroporation method according to protocols as described earlier (Taylor et al., Plasmid; 60:45-52 2008). Transformants were selected on BBL™ Trypticase™ Soy Agar (TSA) (BD Bioscience) plates supplemented with 12.5 μg/mL kanamycin. However, upon multiple successive attempts, few to no colonies were obtained. Restriction-modification (R-M) systems have been known to be wide-spread across the prokaryotic realm (Loenen et al., Nucleic Acids Res.; 42:3-19 2014), yet their presence is poorly predicted in novel genomes and strains. Thus, unique restriction sites present within the Tet_{R}-P_{tetR}-P_{tetA} nucleotide sequence were identified (Table 6). These sites were found in both the TetR (5 times) and P_{tetR}-P_{tetA} (2 times) regions of the nucleotide sequence. The recognition sites present in the TetR coding sequence were changed through the insertion of silent mutations, whereas the recognition sites in the P_{tetR}-P_{tetA} region were changed by making mutations through sticking to the same nucleotide class (purines and pyrimidines). To insert these designed changes into the promoter system, a Gblock was ordered from IDT Technologies and PCR amplified (Table 5), and inserted into pMTL61110Δldh, resulting in pMTL61110-Δldh-Tet_{R}-P_{tetR}-P_{tetA} (mod)-mRuby2 and pMTL61110-Δldh-Tet_{R}-P_{tetR}-P_{tetA}(mod)-BgaB. In these final constructs, the TetR nucleotide sequence was SEQ ID NO:3, the P_{tetR} sequence was SEQ ID NO: 13, the P_{tetA} sequence was SEQ ID NO: 14, the mRuby2 (amino acid) sequence was SEQ ID NO: 15, and the BgaB (amino acid) sequence was SEQ ID NO: 16 (Table 4).

TABLE 5
Overview of primer pairs and templates used in Example 1

| Target name | Primer pair | Template |
|---|---|---|
| Tet_{R}-P_{tetR}-P_{tetA} | 1395_TetR-U-fw | pRPF185 |
| | 1980_tetR(BS)-U-rv | (Fagan and Fairweather, 2011) |
| BgaB | P124_bgaB_U_Fw | pMTLP13B |
| | P125_bgaB_U_Rev | (Ølshøj Jensen et al., AMB |
| | | Express; 7 2017) |
| Tet_{R}-P_{tetR}-P_{tetA}(mod) | P353_TetRnew_UFW | Ordered Gblock (TetR: SEQ ID |
| | P354_TetRnew_URev | NO: 3; P_{tetR}: SEQ ID NO: 13; P_{tetA}: |
| | | SEQ ID NO: 14) |
| LDH upstream homology | PNJ851 | P. thermoglucosidasius |
| region | PNJ852 | DSM2542^{z} gDNA |
| LDH downstream | PNJ853 | P. thermoglucosidasius |
| homology region | PNJ854 | DSM2542^{z} gDNA |
| mRuby2 | P64 | pcDNA3-mRuby2 (Lam et al., |
| | P340 | Nat. Methods 2012) |

Upon removing the seven restriction enzyme recognition sites, transformation of P. thermoglucosidasius DSM2542^{z} was possible and resulted in an increased number of colonies, indicating the importance of removing the recognition sites from the nucleotide sequence. To integrate the Tet_{R}-P_{tetR}-P_{tetA}-mRuby2 or Tet_{R}-P_{tetR}-P_{tetA}-BgaB sequence of the plasmid into the ldh locus of the P. thermoglucosidasius DSM2542^{z} genome, homologous recombination at the desired locus was forced by growing transformants in liquid cultures (2SPY with 12.5 μg/mL kanamycin, 65° C., 200 rotations per minute) and plating on selective solid medium at 65° C. The vector is unable to replicate at this temperature, and so survival only ensues from integration of the complete construct. Colonies were screened for correct integration with colony PCR. To remove the selection marker from the genome, correct integrants were cultured in 2SPY medium at 65° C. for 2-5 days through serial passaging. Double cross-overs were screened through plating on TSA and replica plating on TSA with 12.5 μg/mL kanamycin. Sensitive clones were screened through colony PCR for correct, stable double cross-over and removal of the resistance cassette. The resulting strains created were P. thermoglucosidasius DSM2542^{Z} Δldh::Tet_{R}-P_{tetR}-P_{tetA}-mRuby2 and P. thermoglucosidasius DSM2542^{z} Δldh::Tet_{R}-P_{tetR}-P_{tetA}-BgaB.

Table 6 gives an overview of restriction enzyme recognition sites identified in the Tet_{R}-P_{tetR}-P_{tetA} nucleotide sequence, which were not found in other DNA regions previously known to show efficient transformation into P. thermoglucosidasius strains. The restriction enzymes and known isochimers are shown, as well as the organisms these enzymes have previously been identified in.

TABLE 6
Restriction enzyme recognition sites in the Tet_{R}-P_{tetR}-P_{tetA} nucleotide sequence

| Restriction enzyme | Recognition | | |
|---|---|---|---|
| (and isochimers) | sequence | Previously identified in | SEQ ID NO |
| BsaXI | ACNNNNNCTCC | Bacillus stearothermophilus 25B | 17 |
| AleI, Olil | CACNNNNGTG | Aureobacterium liquefaciens, | 18 |
| | | Oceanospirillum linum 4-5D1 | |
| RleAI | CCCACA | Rhizobium leguminosarum DSM | — |
| | | 5629 | |
| BstENI, EcoNI, | CCTNNNNNAGG | Geobacillus stearoathermophilus | 19 |
| XagI | | EN, Escherichia coli CDC A-193 | |
| | | (ATCC 12041), Xanthobacter | |
| | | agilis Vs18-132 | |
| SacI, Eco53kI, | GAGCTC | Streptomyces achromogenes | — |
| BpuAmI, Ecl136II, | | (ATCC 12767), Escherichia coli | |
| EcolCRI, MxaI, | | 53k, Bacillus pumilus, | |
| Psp124BI, SstI | | Enterobacter cloacae RFL136, | |
| | | Myxococcus xanthus F18E, | |
| | | Pseudomonas sp. 124B, | |
| | | Streptomyces Stanford | |
| BstEZ359I, BstHPI, | GTTAAC | Bacillus stearothermophilus | — |
| HpaI, KspAI, SsrI | | EZ359, Bacillus | |
| | | stearothermophilus HP, | |
| | | Haemophilus parainfluenzae, | |
| | | Kurthia sp. N88, Staphylococcus | |
| | | saprophyticus B6 | |
| BstSNI, Eco105I, | TACGTA | Bacillus stearothermophilus SN, | — |
| SnaBI | | Escherichia coli RFL105, | |
| | | Sphaerotilus natans | |

Initially, the classical, inducer-dependent functioning of the promoter system at 52° C. and 60° C. was investigated. For the evaluation at 52° C., the P. thermoglucosidasius DSM2542^{z} Δldh::Tet_{R}-P_{tetR}-P_{tetA}-mRuby2 strain was used. Single colonies were used to inoculate 5 ml of 2SPY medium, and the cultures were grown for 8 h at 52° C. or 60° C., 200 rotations per minute (RPM). 10 μL of the culture was used to inoculate 50 mL thermophile minimal medium (TMM, as used previously; Pogrebnyakov et al., PLOS One; 12 2017) supplemented with 10 g/L D-Glucose and 0.2% yeast extract (YE). These cultures were left to grow at 52° C. or 60° C., 200 RPM overnight. The following morning, preheated baffled shake flasks with 50 ml TMM, 10 g/l D-Glucose and 0.2% YE were inoculated to a starting optical density (OD) of 0.05 with the overnight culture. The cultures were left to grow at 52° C. or 60° C., 200 RPM, whilst monitoring the OD of the culture. After one duplication of the culture, flasks were induced by addition of anhydrotetracycline (stock solution at 2 g/L) to a final concentration of 0.5 μg/mL. No anhydrotetracycline was added to uninduced control cultures. For the following 10 hours post induction, the OD was monitored, and 1 mL samples were taken. Cells from the sample were fixed by incubation for 30-60 min in 2% paraformaldehyde in phosphate-buffered saline (PBS).

After incubation, cells were washed with and stored in PBS at 4° C. for up to 2 weeks. The fluorescence of the fixed cells was determined with a MACSQuant VYB cytometer (Miltenyi Biotec, Germany). mRuby2 fluorescence was measured by excitation with a 561 nm laser, using a 615/20 nm band-pass filter. Per 200 μL sample, up to 50,000 events were recorded. Gain settings used were; forward scatter: 580 V, side scatter: 516 V, Y2 channel: 492 V. Data was analyzed using FlowJo (Tree Star Inc., Asland, OR).

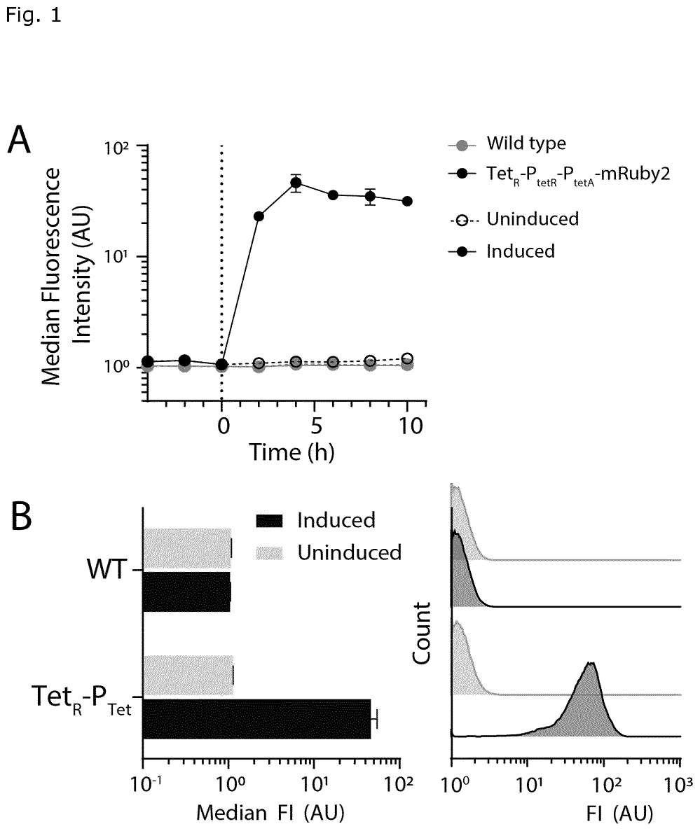

As shown in FIG. 1A, the mRuby2 expression from Tet_{R}-P_{tetR}-P_{tetA} when uninduced at 52° C. is indistinguishable from the wild type (WT) control, whereas induction drastically increases mRuby2 expression and does so in a homogenous manner as seen through the flow histograms (FIG. 1B). The signal is also stable for a period of at least 8 hours post induction (FIG. 1A). This shows that, unexpectedly, the expression system has a similar function to the way it behaves in mesophilic hosts, even at the 15° C. higher temperature.

Next, we characterized the function of the Tet_{R}-P_{tetR}-P_{tetA} expression system at 60° C., through classical induction, as described above, with the BgaB activity as output. BgaB activity was assayed based on previously described protocols (Zhang et al., J. Biol. Chem.; 270:11181-11189 1995), but the concentration of dibasic sodium phosphate in the permeabilization solution was lowered to 100 mM. Activity was measured at 60° C., and the reaction stopped after 20 minutes. The absorbance (A₄₂₀ₙₘ and A₆₀₀ₙₘ) of 200 μL of the reaction supernatant was measured in a 96-well microtiter plate. The Miller units gives a readout for BgaB activity and was calculated according to Formula I:

$$Miller units = 1000 * \frac{{A}_{420 n m}}{( {A}_{600 nm} * V * t )} \qquad ( I )$$

where the volume of the sample used (V, 20 μL) and exact assay reaction time (t) are also used. Directly upon taking the sample, 20 μL was added to the permeabilization solution and stored at 4° C. overnight, until the assay was run with all samples simultaneously. Previous experiments indicated this did not influence the BgaB activity when compared to freshly taken samples.

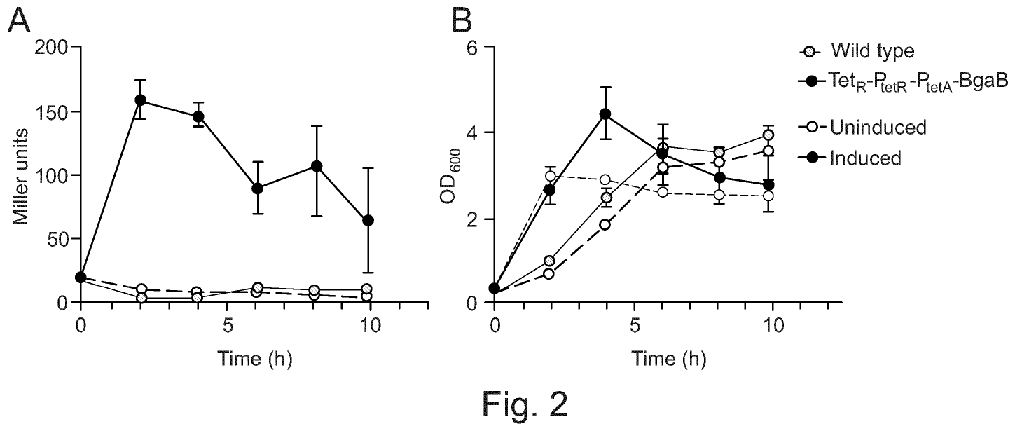

It is seen that at 60° C., the Tet_{R}-P_{tetR}-P_{tetA} expression system still functions as it does at lower temperatures, being highly inducible yet extremely silent when uninduced (FIG. 2A). However, the time stability of the system appears different, though this may be an artifact resulting from the sporulation observed to occur after 4 hours post induction, due to the test being performed in batch culture (FIG. 2B).

Finally, to investigate the temperature inducibility of the expression system, single colonies were used to inoculate 5 mL of 2SPY medium and grown for 8 h at 60° C., 200 rotations per minute (RPM). 10 μL of the culture was used to inoculate 50 mL thermophile minimal medium (TMM, as used previously; Pogrebnyakov et al., PLOS One;12 2017) supplemented with 10 g/L D-Glucose and 0.2% yeast extract. These cultures were left to grow at 60° C., 200 RPM overnight. The following morning, preheated shake flasks with 50 mL TMM, 10 g/L D-Glucose and 0.2% YE and a magnetic stirrer rod were inoculated to a starting OD of 0.05 with the overnight culture. The cultures were left to grow at 60° C., 200 RPM, whilst monitoring the OD of the culture. After one duplication of the culture, flasks were induced by transfer to a preheated, temperature-controlled water bath set to 65° C. placed on a magnetic stirrer plate. Uninduced control flasks were left at 60° C. For the following 8 hours post induction, the OD was monitored, and samples of the culture were taken every two hours to quantify BgaB expression.

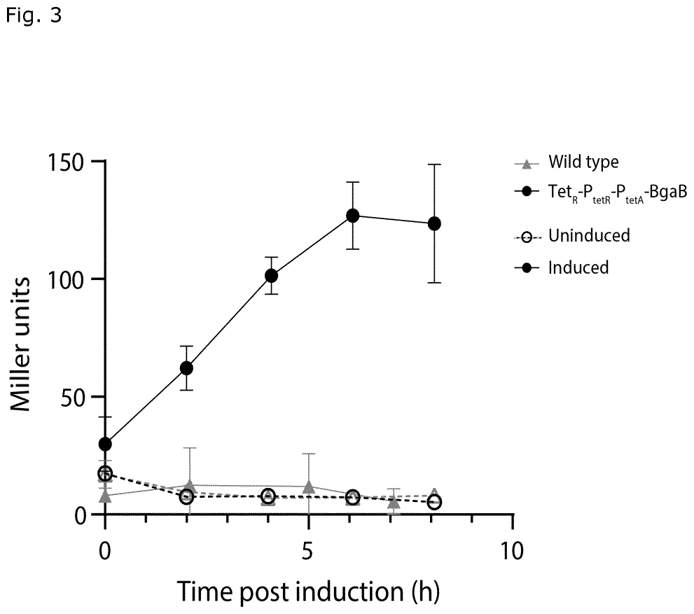

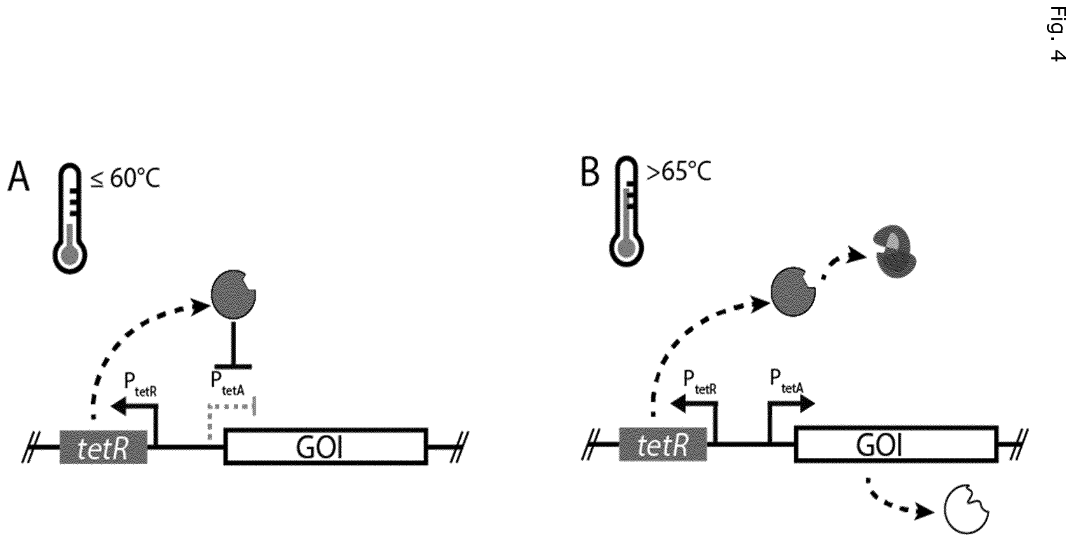

As shown in FIG. 3, at 60° C. the BgaB expression from Tet_{R}-P_{tetR}-P_{tetA} is very silent and indistinguishable from the wild type background signal. This indicates the Tet_{R}-P_{tetR}-P_{tetA}functions similarly at 60° C. as it does at lower, mesophilic temperatures. Surprisingly, upon shifting culture temperature to 65° C., induction of the BgaB construct from the Tet_{R}-P_{tetR}-P_{tetA}promoter was observed within two hours, independent of the presence or absence of inducer. Maximal induction was reached after 6 hours post induction and was maintained until the maximum sample point of 8 hours post induction. These data demonstrate that the Tet_{R}-P_{tetR}-P_{tetA} system can be used by simply shifting culture temperature and thereby induce expression of target proteins (see schematic representation in FIG. 4).

TABLE 7
Primer sequences

| | | SEQ ID |
|---|---|---|
| Name | Sequence | NO |
| P353_TetRnew_UFW | ACAATUTCACACAGGAGGCCGA | 20 |
| P354_TetRnew_URev | ATCGCCUCAGCTTACTGCAGGAGGAC | 21 |
| 1395_TetR-U-fw | atcgcgU ttaagacccactttcacatttaagttgt | 22 |
| 1980_tetR(BS)-U-rv | atcgccUcagc ttactgcaggagctcagatctgt | 23 |
| P124_bgaB_U_Fw | GGCGAUaggaggaatataccATGAACGTTTTATCCTCAATTTG | 24 |
| P125_bgaB_U_Rev | AGAGTUAGAAAAAGAAACAGAGGCTACTCTCAA | 25 |
| PNJ851 | ACGAATUCCCTCGGCAAACAGAGCTTTAAAACC | 26 |
| PNJ852 | AACAAGGUGAACATCGTGTGGATACAAC | 27 |
| PNJ853 | AACTCUGCCATTATCATTTCCGTAATGCC | 28 |
| PNJ854 | ATCCCCGGGUACGGACTTTTATTTCAATTCGTATGGTGCG | 29 |
| P64 | GGCGAUaggaggaatatacatggtgtctaagggcgaagag | 30 |
| P340 | GGTGCGAUTTACTTGTACAGCTCGTC | 31 |

### EXAMPLE 2

### Simulation of Temperature-Dependent Stability of TetR Family Members

This example demonstrates an in silico simulation of the temperature-dependent (in) stability of members of the TetR family. The results of this simulation indicate that TetR proteins in general, irrespective of whether they are derived from mesophilic, thermophilic or psychrophilic hosts, are amenable to be used for control of gene expression by temperature regulation in the thermophilic temperature range.

As shown in Example 1, upon temperature increase, the TetR protein loses its capacity to bind to DNA and repress expression from the Pₜₑₜ promoter. To identify possible structural and sequence components of TetR that are causative for the structural change upon temperature increase, we deployed molecular dynamic (MD) simulations. MD simulations predict the physical movements of atoms and molecules and is a benchmark for analyzing protein stability under changing conditions (Karplus and McCammon, Nat. Struct. Biol.; 9:646-652 2002). From this, flexible regions in the protein can be identified, which are often unstable at the tested conditions.

The flexibility of TetR was modeled at low and high temperatures by rapid, large scale molecular dynamics (MD) simulation and analysis using the online version of the coarse-grained modeling tool CABS-flex 2.0 (Kuriata et al., Nucleic Acids Res.; 46: W338-W343 2018).

The atomic coordinates for TetR was obtained from the PDB ID 2XPW. The PDB file was manually inspected using PyMOL version 2.5.4 (Schrodinger and LLC, 2015) to select the chains corresponding to the dimer whose solved structure covered most of the protein sequence. Small missing N- or C-terminal segments were not modelled, as they were expected to have negligible influence on the simulation results. PyMOL was also used to fix missing chain IDs in the PDB file when needed.

To simulate an entire dimer, two chains corresponding to a monomer each were selected for the PDB file. The standard distance restraint values suggested by CABS-flex 2.0 were used, i.e., the mode was SS2, with a restrain gap along the chain of 3 residues, and the minimal and maximal length of restraint were 3.8 and 8.0 Å, respectively. No additional distance restraints were added. Independent simulations were performed at low (1.4) and high (2.0) temperatures.

The main results generated by CABS-flex 2.0 (publicly available at the internet webpage http://biocomp.chem.uw.edu.pl/CABSflex2/index, accessed on 6 Dec. 2022), are conformational models, contact maps, and fluctuation plots, and our analysis focused on the latter. Since the monomers forming a dimer in a simulation are identical, they were treated as replicates of each other and hence their root mean square fluctuations (RMSFs) were averaged for each amino acid residue.

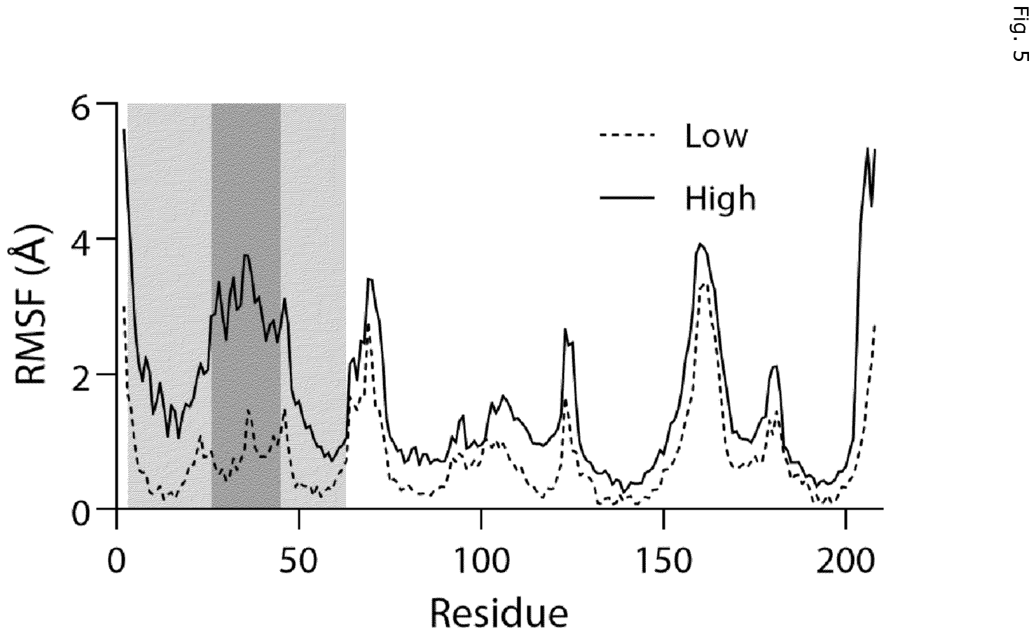

For TetR, the MD simulations reveal regions showing a higher level of increased flexibility upon increased temperature (FIG. 5). Of these three regions, two are found at the (generally flexible) termini of the protein. The final region identified falls in the conserved helix-turn-helix (HTH) region of the protein, even more specifically in the known DNA-binding domain. Therefore, at higher temperature, the MD simulations predict a loss of structure in the DNA-binding domain, which can result in a loss of affinity for the tetO sequence, and so trigger expression from Pₜₑₜ.

### Conservation of Functionality Across the TetR Family

The TetR family is classified based on the presence of the highly conserved HTH region, often found near the N-terminus of the protein. The HTH region forms the basis for the DNA-binding domain of the protein, and the residual 70-75% of the protein can dictate alternative functional parameters, such as inducer specificity. The highly conserved nature of the HTH-region results in proteins of the TetR family being found in nearly all sequenced bacteria to date, even though they have very different response elements and inducer specificities (Stanton et al., Nat. Chem. Biol.; 10:99-105 2014).

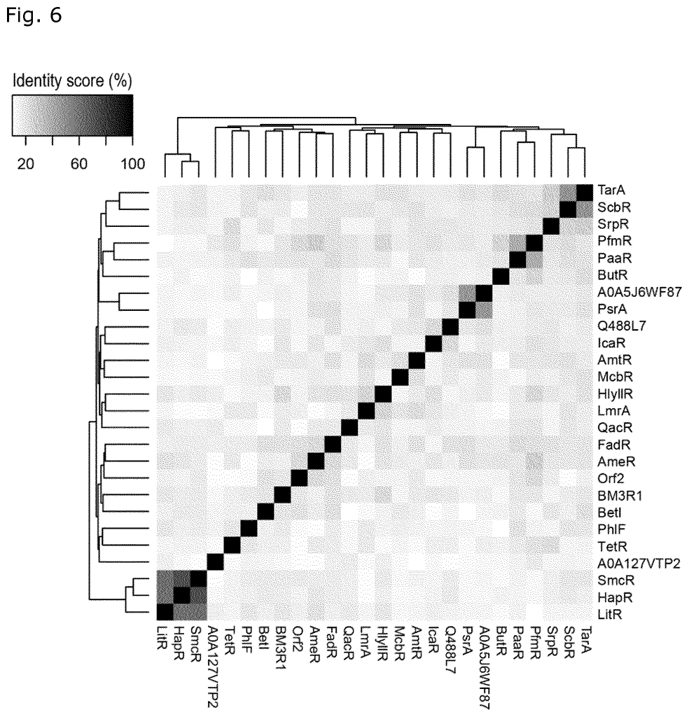

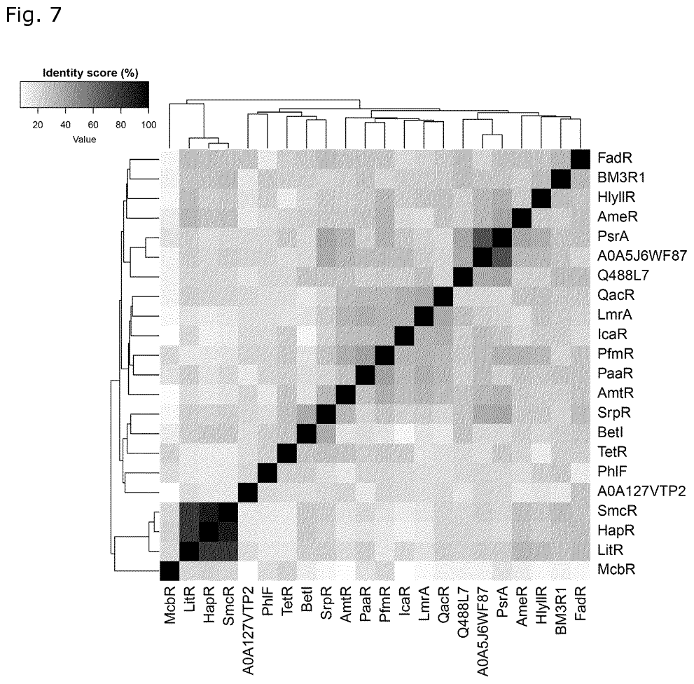

To investigate the level of conservation across the TetR family, in relation to potential for temperature-based induction, a Multiple Alignment using Fast Fourier Transform (MAFFT) was run on a subset of TetR family proteins (Madeira et al., Nucleic Acids Res.; 47: W636-W641 2019). The variants with identified inducers and operator sites from Stanton et al. were selected, as well as three variants from thermophilic and psychrophilic (optimum growth <20° C.) hosts (Table 8; Agari et al., Microbiology; 157:1589-1601 2011). MAFFT analysis on the whole protein structure as well as on the conserved HTH-domain was performed (FIGS. 6 and 7, respectively). The sequences used for performing the MAFFT can be found in Tables 9 and 10, respectively. This analysis shows how diverse the TetR family proteins are, both when looking at whole protein sequence and the HTH sequence. Even though the HTH-domain has higher similarity compared to the whole protein analysis, there is still variation in amino acid sequence. Though this is observed on amino acid level, the 3D structure and hence function of the HTH motif is still highly conserved within the family.

Using the crystal structure of the identified variants, the melting temperature can be estimated using predictive algorithms (Table 8; Pucci et al., Bioinformatics; 33:3415-3422 2017). For TetR, the crystal structure reveals a predicted melting temperature of 60.5° C., close to what was observed for the in vitro data presented in Example 1. Interestingly, for all the variants tested, regardless of their organism source (i.e. psychrophilic, mesophilic or thermophilic), their melting temperature falls in the thermophilic operating range.

As the conserved DNA-binding domain was shown to be potentially causative for the TetR temperature induction (FIG. 5), its role in regulating potential temperature inducible systems in the other TetR family proteins was investigated in the same manner. The PDB IDs for the analyzed proteins are shown in Table 8. In the case of IcaR, where all PDBs presented unresolved loops in the internal part of the sequence, the online tool ModLoop (Fiser et al., Protein Sci.; 9:1753-1773 2000; Fiser and Sali, Bioinformatics; 19:2500-2501 2003).

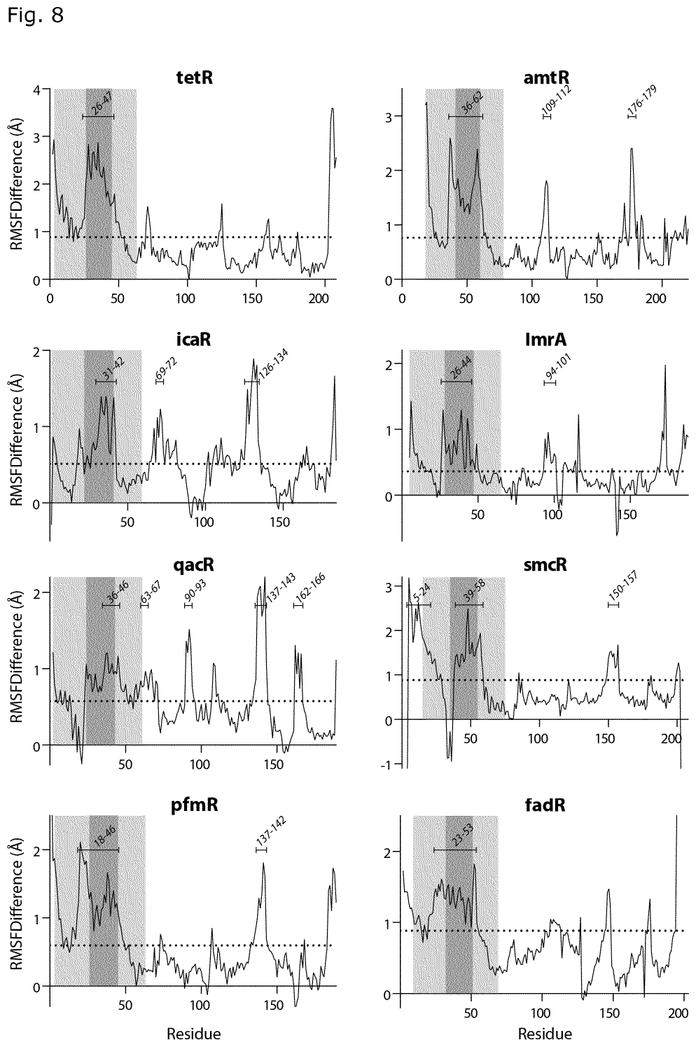

By performing the same MD simulations, the difference in RMSF values (ΔRMSF) for each residue between low and high temperature can be calculated. From there, destabilized regions are defined as regions where the ΔRMSF for a residue is higher than 1.5 times the overall average RMSF value for the whole protein (ΔRMSF>1.5x̅_{ΔRMSF}) for at least four consecutive residues. This analysis was performed for a representative subset of the TetR family proteins (FIG. 8). Overlaying the destabilized regions with the HTH region and DNA-binding domains shows that every TetR-family protein analyzed has a predicted destabilization region within the HTH- and DNA binding regions. Some contain additional destabilization regions in other parts of the protein. This points to the possibility that temperature-based induction of the TetR proteins in the thermophilic range may be feasible across the TetR family.

TABLE 8
TetR family proteins used to analyze sequence similarity and for further
MD simulations. The growth preference of the host from which the protein
was found is indicated. Percentage identity to the whole TetR protein is
indicated, when analyzed by MAFFT. The PDB ID for the crystal structure
of the protein or Alpha Fold ID for the predicted structure is provided.
Melting temperature predicted based on protein structure as calculated
by SCooP is shown (Pucci et al., Bioinformatics; 33: 3415-3422 2017).

| | | % AA identity to | | Predicted Tₘ |
|---|---|---|---|---|
| Protein | Origin | whole tetR | PDB or AlphaFold ID | (° C.) |
| TetR | Mesophilic | 100.00 | 1QPI | 60.5 |
| ImrA | Mesophilic | 14.77 | 1SGM | 59.8 |
| AmtR | Mesophilic | 15.70 | 5MQQ | 62.2 |
| SrpR | Mesophilic | 16.67 | AF-Q9R9T9-F1 | 57.4 |
| TarA | Mesophilic | 14.62 | NA | |
| QacR | Mesophilic | 15.12 | 1JT0 | 68 |
| IcaR | Mesophilic | 19.38 | 2ZCN | 67.9 |
| ScbR | Mesophilic | 14.88 | NA | |
| ButR | Mesophilic | 15.05 | NA | |
| PhlF | Mesophilic | 17.86 | AF-Q9RF02-F1 | 60.8 |
| SmcR | Mesophilic | 15.08 | 3KZ9 | 58.7 |
| McbR | Mesophilic | 14.61 | 4P9F | 59.5 |
| PsrA | Mesophilic | 13.19 | 2FBQ | 61.4 |
| BetI | Mesophilic | 19.88 | AF-P17446-F1 | 62.6 |
| LitR | Mesophilic | 14.86 | AF-Q8KX64-F1 | 54.6 |
| Orf2 | Mesophilic | 11.24 | NA | |
| HapR | Mesophilic | 14.12 | 2PBX | 58.1 |
| HlyllR | Mesophilic | 12.29 | 2FX0 | 65.9 |
| AmeR | Mesophilic | 16.28 | AF-Q7CWT2-F1 | 56.3 |
| BM3R1 | Mesophilic | 18.54 | AF-P43506-F1 | 63.3 |
| Q488L7 | Psychrophilic | 15.66 | AF-Q488L7-F1 | 46.9 |
| A0A127VTP2 | Psychrophilic | 12.50 | AF-A0A127VTP2-F1 | 54.7 |
| A0A5J6WF87 | Psychrophilic | 15.56 | AF-A0A5J6WF87-F1 | 50.0 |
| FadR | Thermophilic | 14.04 | 3ANP | 71.6 |
| PaaR | Thermophilic | 14.81 | AF-Q5SJN5-F1 | 81.9 |
| PfmR | Thermophilic | 17.14 | 3VPR | 87.5 |

NA = Not available.

TABLE 9
SEQ ID NOs of amino acid sequences used in the analyses shown in FIGS. 6 and 7

| | | SEQ ID NO of amino acid | SEQ ID NO of amino acid |
|---|---|---|---|
| Protein | Origin | sequence used in FIG. 6 | sequence used in FIG. 7 |
| TetR | Mesophilic | 1 | 57 |
| ImrA | Mesophilic | 32 | 58 |
| AmtR | Mesophilic | 33 | 59 |
| SrpR | Mesophilic | 34 | 60 |
| TarA | Mesophilic | 35 | — |
| QacR | Mesophilic | 36 | 61 |
| IcaR | Mesophilic | 37 | 62 |
| ScbR | Mesophilic | 38 | — |
| ButR | Mesophilic | 39 | — |
| PhlF | Mesophilic | 40 | 63 |
| SmcR | Mesophilic | 41 | 64 |
| McbR | Mesophilic | 42 | 65 |
| PsrA | Mesophilic | 43 | 66 |
| BetI | Mesophilic | 44 | 67 |
| LitR | Mesophilic | 45 | 68 |
| Orf2 | Mesophilic | 46 | — |
| HapR | Mesophilic | 47 | 69 |
| HlyllR | Mesophilic | 48 | 70 |
| AmeR | Mesophilic | 49 | 71 |
| BM3R1 | Mesophilic | 50 | 72 |
| PfmR | Thermophilic | 51 | 73 |
| PaaR | Thermophilic | 52 | 74 |
| FadR | Thermophilic | 53 | 75 |
| Q488L7 | Psychrophilic | 54 | 76 |
| A0A127VTP2 | Psychrophilic | 55 | 77 |
| A0A5J6WF87 | Psychrophilic | 56 | 78 |

### LIST OF REFERENCES

Each reference listed below or described elsewhere herein is hereby incorporated by reference in its entirety.
Agari, Y., Agari, K., Sakamoto, K., Kuramitsu, S., abd Shinkai, A. (2011). TetR-family transcriptional repressor Thermus thermophilus FadR controls fatty acid degradation. Microbiology. 157:1589-1601.
Bertram, R., and Hillen, W. (2008). The application of Tet repressor in prokaryotic gene regulation and expression. Microb. Biotechnol. 1 (1): 2-16. doi: 10.1111/j.1751-7915.2007.00001.x
Corrigan, R. M., and Foster, T. J. (2009). An improved tetracycline-inducible expression vector for Staphylococcus aureus. Plasmid. 61 (2): 126-129. doi: 10.1016/j.plasmid.2008.10.001.
Cuthbertson, L., and Nodwell, J. R. (2013). The TetR family of regulators. Microbiology and Molecular Biology Reviews. 77 (3). doi: 10.1128/MMBR.00018-13.
He, P., et al. (2017). High-level production of a-amylase by manipulating the expression of alanine racamase in Bacillus licheniformis. Biotechnol. Lett. 39:1389-1394. doi: 10.1007/s10529-017-2359-5
Holland, A. T. N., et al. (2019). Inhibition of extracellular proteases improves the production of a xylanase in Parageobacillus thermoglucosidasius. BMC Biotechnol. 19. doi: 10.1186/s12896-019-0511-0
Ettner, N., et al. (1996). Fast large-scale purification of tetracycline repressor variants from overproducing Escherichia coli strains. Journal of Chromatography A, 742:95-105.
Fagan and Fairweather (2011). Clostridium difficile has two parallel and essential Sec secretion systems. J. Biol. Chem. 286:27483-27493.
Fiser, A., Do, R. K. G., and Sali, A. (2000). ModLoop: Automated modeling of loops in protein structures. Protein Sci. 9:1753-1773.
Fiser, A., and Sali, A. (2003). ModLoop: Automated modeling of loops in protein structures. Bioinformatics. 19:2500-2501.
Ito, A., et al. (2012). Heat-inducible transgene expression with transcriptional amplification mediated by a Int. transactivator. J. Hypertherm. 28:788-798. doi: 10.3109/02656736.2012.738847
Kamionka, A., et al. (2005). Tetracycline-dependent conditional gene knockout in Bacillus subtilis. Appl. Environ. Microbiol. 71 (2): 728-733. doi: 10.1128/AEM.71.2.728-733.2005.
Kamionka, A., et al. (2004). Two mutations in the tetracycline repressor change the inducer anhydrotetracycline to a corepressor. Nucleic Acids Res. 32:842-847.
Karplus, M., and McCammon, J. A. (2002). Molecular dynamics simulations of biomolecules. Nat. Struct. Biol. 9:646-652.
Krueger, C., et al. (2018). Tetracycline derivatives: alternative effectors for Tet transregulators. Biotechniques. 37 (4). doi: 10.2144/04374BM04
Kuriata, A., et al. (2018). CABS-flex 2.0: A web server for fast simulations of flexibility of protein structures. Nucleic Acids Res. 46: W338-W343.
Lam, A.J., et al. (2012). Improving FRET dynamic range with bright green and red fluorescent proteins. Nat. Methods. 9:1005-1012. doi: 10.1038/nmeth.2171. 10.1038/nmeth.2171
Lin, P., et al. (2014). Isobutanol production at elevated temperatures in thermophilic Geobacillus thermoglucosidasius. Metabol. Engineer. 24:1-8.doi: 10.1016/j.ymben.2014.03.006.
Madeira, F., et al. (2019). The EMBL-EBI search and sequence analysis tools APIs in 2019. Nucleic Acids Res. 47: W636-W641.
Menart, V., et al. (2003). Constitutive versus thermoinducible expression of heterologous proteins in Escherichia coli based on strong PR,PL promoters from phage lambda. Biotechnol. Bioeng. 83:181-190. doi: 10.1002/bit.10660.
Müller, G., et al. (1995). Characterization of non-inducible Tet repressor mutants suggests conformational changes necessary for induction. Nature Structural Biology 2:693-703.
Orth, P., et al. (2000). Structural basis of gene regulation by the tetracycline inducible Tet repressor-operator system. Nature Structural Biology. 7 (3): 215-219.
Pogrebnyakov, I., et al. (2017). Genetic toolbox for controlled expression of functional proteins in Geobacillus spp. PLOS ONE. 12 (2): e0171313. doi: 10.1371/journal.pone.0171313
Pucci, F., Kwasigroch, J. M., and Rooman, M. (2017). SCooP: an accurate and fast predictor of protein stability curves as a function of temperature. Bioinformatics. 33:3415-3422.
Ramos, J. L., et al. (2005). The TetR family of transcriptional repressors. Microbiology and Molecular Biology Reviews. 69 (2): 326-356.
Reichheld, S. E., et al. (2009). The induction of folding cooperativity by ligand binding drives the allosteric response of tetracycline repressor. PNAS. 106 (52): 22263-22268. doi: 10.1073/pnas.0911566106
Scholz, O., et al. (2004). Activity reversal of Tet repressor caused by single amino acid exchanges. Mol. Microbiol. 53:777-789.
Schrödinger and LLC. (2015). PyMOL molecular graphics system.
Sheng, L., et al. (2017). Development and implementation of rapid metabolic engineering tools for chemical and fuel production in Geobacillus thermoglucosidasius NCIMB 11955. Biotechnol. Biofuels. 10:1-18.
Sizemore, C., et al. (1990). Quantitative analysis of Tn10 Tet repressor binding to a complete set of tet operator mutants. Nucleic Acids Res. 18 (10): 2875-2880.
Stanton, B. C., et al. (2014). Genomic mining of prokaryotic repressors for orthogonal logic gates. Nat. Chem. Biol. 10:99-105.
Styles, M. Q., et al. (2021). The heterologous production of terpenes by the thermophile Parageobacillus thermoglucosidasius in a consolidated bioprocess using waste bread. Metabol. Engineer. 65:146-155. doi: 10.1016/j.ymben.2020.11.005
Valdez-Cruz, N.A., et al. (2010). Production of recombinant proteins in E. coli by the heat inducible expression system based on the phage lambda pL and/or pR promoters. Microb. Cell Fact. 9:18. doi: 10.1186/1475-2859-9-18
Vasina, J. A., et al. (1998). Scale-up and optimization of the low-temperature inducible cspA promoter system. Biotechnol. Prog. 14:714-721. doi: 10.1021/bp980061p.
Wickstrum, J., et al. (2013). Conditional gene expression in Chlamydia trachomatis using the Tet system. PLOS ONE. 8 (10): e76743. doi: 10.1371/journal.pone.0076743
Yang, Z., et al. (2020). Engineering thermophilic Geobacillus thermoglucosidasius for riboflavin production. Microb. Biotechnol. doi: 10.1111/1751-7915.13543
Zhang, X., et al. (1995). Control of the Escherichia coli rrnB P1 promoter strength by ppGpp. J. Biol. Chem. 270 (19): 11181-11189.
Zhou, J., Lian, et al. (2020). Metabolic engineering of Parageobacillus thermoglucosidasius for the efficient production of (2R, 3R)-butanediol. Appl. Microbiol. Biotechnol. 104:4303-4311. doi: 10.1007/s00253-020-10553-8
WO2007074991 A1 (CJ Corp.)
WO17147555 A1 (Lanzatech New Zealand Ltd.)
WO16060714 A1 (American Sterilizer Co.)
WO13144647 A1 (Univ. Nottingham)
WO08079469 A2 (American Sterilizer Company)
US2006121564 A1 (National Institute of Advanced Industrial Science and Technology, Tokyo)
US2009042301 A1 (Univ. Alberta)
U.S. Pat. No. 10,745,693 B2 (Wisconsin Alumni Res. Found.)
U.S. Pat. No. 4,874,703 A (Eli Lilly and Company)
EP0685560 A2 (Suntory Ltd.)
CN105838716 A (Univ. Southwest)
CN110066758 A (Wuhan Junan Biological Tech Co Ltd.)

1. A thermophilic host cell comprising a heterologous expression control system comprising a Tet repressor (TetR) protein and a first promoter comprising a tet operator (tetO) sequence to which the TetR protein can bind, wherein the first promoter is operatively linked to at least one gene of interest.

2. Use of an expression control system comprising a TetR protein and a first promoter comprising a tetO sequence to which the TetR protein can bind to control the expression of at least one gene of interest in a thermophilic host cell by temperature regulation.

3. The thermophilic host cell according to claim 1, wherein the thermophilic host cell further comprises a gene encoding the TetR protein.

4. The thermophilic host cell according to claim 1, wherein,
(a) when the thermophilic host cell is incubated in a culture medium having a first temperature, the expression of the gene of interest is not induced;
(b) when the thermophilic host cell is incubated in a culture medium having a second temperature, the expression of the gene of interest is induced; and
(c) the second temperature is at least about 4° C. higher than the first temperature.

5. A method of controlling the expression of at least one gene of interest in a thermophilic host cell comprising an expression control system comprising a TetR protein and a first promoter comprising a tetO sequence, wherein the first promoter is operatively linked to the at least one gene of interest, the method comprising:
(i) incubating the thermophilic host cell in a culture medium having a first temperature; and
(ii) incubating the thermophilic host cell in a culture medium having a second temperature which is at least about 4° C. higher than the first temperature, wherein steps i) and ii) can be conducted in any order,
wherein the expression of the at least one gene of interest is induced at the second temperature but not at the first temperature, thereby controlling the expression of the at least one gene of interest.

6. A method of producing a gene product of interest in a thermophilic host cell comprising an expression control system comprising a TetR protein and a first promoter comprising a tetO sequence to which the TetR protein can bind, wherein the first promoter is operatively linked to a gene of interest encoding the gene product of interest, the method comprising:
(i) incubating the thermophilic host cell in a culture medium having a first temperature; and
(ii) incubating the thermophilic host cell in a culture medium having a second temperature which is at least about 4° C. higher than the first temperature; and
wherein the expression of the gene encoding the gene product of interest is induced at the second temperature but not at the first temperature, thereby producing the gene product of interest.

7. The method according to claim 5, comprising a prior step of transforming or transfecting the thermophilic host cell with one or more nucleic acid constructs comprising the first promoter, the at least one gene of interest and a gene encoding the TetR protein.

8. The thermophilic host cell according to claim 4, wherein the medium has not been supplemented with tetracycline or an analog thereof which binds to the TetR protein.

9. The thermophilic host cell according to claim 4, wherein the first temperature is at most about 60° C., the second temperature is at least about 65° C., or both.

10. The thermophilic host cell according to claim 1, wherein,
(a) when the TetR protein is bound to the tetO sequence of the first promoter, the promoter is repressed; and
(b) when the TetR protein is not bound to the tetO sequence of the first promoter, the promoter is active.

11. The thermophilic host cell according to claim 1, wherein the TetR protein
a) comprises an amino acid sequence at least 60% identical to a sequence selected from SEQ ID NOs: 1, 32-56, and 79-82,
b) has a melting temperature between 40° C. and 90° C.,
c) comprises a helix-turn-helix (HTH) region, which comprises a DNA-binding domain and a sub-region of at least 4 consecutive amino acid residues, which sub-region destabilizes upon temperature increase, or
(d) a combination of (a) and (b), (a) and (c) or all of (a) to (c).

12. The thermophilic host cell according to claim 1, wherein
(a) the TetR protein comprises an amino acid sequence at least 60% identical to SEQ ID NO:1; and/or
(b) the tetO sequence comprises a nucleotide sequence at least 65% identical to SEQ ID NO:5.

13. The thermophilic host cell according to claim 1, wherein the expression control system comprises a TetR protein comprising an amino acid sequence at least 90% identical to SEQ ID NO: 1, a tetO nucleotide sequence at least 90% identical to SEQ ID NO:5, and a first promoter comprising a nucleotide sequence at least 90% identical to SEQ ID NO:14.

14. The thermophilic host cell according to claim 1, wherein the thermophilic host cell has an optimum growth temperature in the range of about 55° C. to about 65° C.

15. The thermophilic host cell according to claim 1, wherein the thermophilic host cell is a bacterium.

16. The thermophilic host cell according to claim 15, wherein the bacterium is selected from the group consisting of: Parageobacillus thermoglucosidasius, Geobacillus toebii, Geobacillus stearothermophilus, Geobacillus thermodenitrificans, Geobacillus kaustophilus, Geobacillus thermoleovorans, Geobacillus thermocatenulatus, Thermoanaerobacterium xylanolyticum, Thermoanaerobacterium saccharotyticum, Thermoanaerobacterium thermosaccharolyticum, Thermoanaerobacter mathranii, Thermoanaerobacter pseudoethanolicus, Thermoanaerobacter brockii, Thermoanaerobacter kivui, Thermoanaerobacter brockii, Caldanaerobacter subterraneus, Clostridium thermocellum, Clostridium thermosuccinogenes, Thermoclostridium stercorarium, Bacillus licheniformis, Bacillus coagulans, Bacillus smithii, Bacillus methanolicus, Bacillus flavothermus, Anoxybacillus kamchatkensis, Anoxybacillus gonensis, Caldicellulosiruptor bescii, Caldicellulosiruptor saccharolyticus, Caldicellulosiruptor kristjanssonii, Caldicellulosiruptor owensensis, Caldicellulosiruptor lactoaceticus, Moorella thermoacetica, Moorella thermoautotrophica, Thermus thermophilus, Thermus aquaticus, Thermotoga maritima, Pseudothermotoga lettingae, Pseudothermotoga thermarum, Chloroflexus aurantiacus, Anaerocellum thermophilum, Rhodothermus marinus, Sulfolobus acidocaldarius, Sulfolobus islandicus, Sulfolobus solfataricus, Thermococcus barophilus, Thermococcus kodakarensis, Pyrococcus abyssi, and Pyrococcus furiosus.

17. The thermophilic host cell according to claim 3, wherein the gene encoding the TetR protein is operatively linked to a second promoter.

18. The method according to claim 6, comprising a step (iii) of isolating the gene product, after step (ii).

19. The method according to claim 7, wherein the gene encoding the TetR protein is operatively linked to a second promoter.

20. The thermophilic host cell according to claim 11, wherein the TetR protein has a melting temperature between 55° C. and 90° C.

21. The thermophilic host cell according to claim 20, wherein the TetR protein has a melting temperature between 60° C. and 90° C.

22. The thermophilic host cell according to claim 13, wherein the expression control system further comprises a second promoter comprising a nucleic acid sequence at least 90% identical to SEQ ID NO:13.

23. The thermophilic host cell according to claim 14, wherein the thermophilic host cell has an optimum growth temperature at about 60° C.


SEQUENCE LISTING (82 sequences)

SEQ ID NO: 1 - protein, 207 aa, Escherichia coli
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 2 - DNA, 627 bp, Escherichia coli
  source 1..627: mol_type = genomic DNA
atgatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaa

SEQ ID NO: 3 - DNA, 627 bp, synthetic construct
  misc_feature 1..627: note = Recombinant sequence
  source 1..627: mol_type = other DNA
atgatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgcccattagaaggggaaagctggcaagattttttgagaaataacgctaaaagttttagatgtgctttactaagtcatcgcgatggggcaaaagtacatttaggtacacggcctacagaaaaacagtatgaaactctcgaaaatcaattagcctttttatgccaacaaggtttttcactagagaatgcattatatgcactcagcgctgttggtcattttactttaggttgcgtattggaagatcaagagcatcaagtcgctaaagaagaaagggaaacacctactactgatagtatgccgccattattacgacaagctatcgaattatttgatcatcaaggggcagagccagccttcttattcggccttgaattgatcatatgcggattagaaaaacaacttaaatgtgaaagtgggtcttaa

SEQ ID NO: 4 - DNA, 19 bp, Escherichia coli
  source 1..19: mol_type = genomic DNA
actctatcaatgatagagt

SEQ ID NO: 5 - DNA, 19 bp, synthetic construct
  misc_feature 1..19: note = Recombinant sequence
  source 1..19: mol_type = other DNA
actctatcattgatagagt

SEQ ID NO: 6 - DNA, 19 bp, synthetic construct
  misc_feature 1..19: note = Recombinant sequence
  source 1..19: mol_type = other DNA
tccctatcagtgatagaga

SEQ ID NO: 7 - DNA, 19 bp, synthetic construct
  misc_feature 1..19: note = Recombinant sequence
  source 1..19: mol_type = other DNA
tccctaacagtgttagaga

SEQ ID NO: 8 - DNA, 19 bp, synthetic construct
  misc_feature 1..19: note = Recombinant sequence
  source 1..19: mol_type = other DNA
tccctttcagtgaaagaga

SEQ ID NO: 9 - DNA, 19 bp, synthetic construct
  misc_feature 1..19: note = Recombinant sequence
  source 1..19: mol_type = other DNA
tcccaatcagtgattgaga

SEQ ID NO: 10 - DNA, 19 bp, synthetic construct
  misc_feature 1..19: note = Recombinant sequence
  source 1..19: mol_type = other DNA
tccgtatcagtgatacaga

SEQ ID NO: 11 - DNA, 19 bp, synthetic construct
  misc_feature 1..19: note = Recombinant sequence
  source 1..19: mol_type = other DNA
tccccatcagtgatggaga

SEQ ID NO: 12 - DNA, 81 bp, synthetic construct
  misc_feature 1..81: note = Recombinant sequence
  source 1..81: mol_type = other DNA
ttgacactctatcattgatagagtataattaaaataagcttgatcgtagcgttaacagatctgagctcctgcagtaagctg

SEQ ID NO: 13 - DNA, 49 bp, synthetic construct
  misc_feature 1..49: note = Recombinant sequence
  source 1..49: mol_type = other DNA
tgacaaataactctatcaatgatataatgtcaacaaaaaggaggaatta

SEQ ID NO: 14 - DNA, 81 bp, synthetic construct
  misc_feature 1..81: note = Recombinant sequence
  source 1..81: mol_type = other DNA
ttgacactctatcattgatagagtataattaaaataagcttgatcgtagcgattacagatctgtcctcctgcagtaagctg

SEQ ID NO: 15 - protein, 181 aa, synthetic construct
  REGION 1..181: note = Recombinant sequence
MVSKGEELIKENMRMKVVMEGSVNGHQFKCTGEGEGNPYMGTQTMRIKVIEGGPLPFAFDILATSFMYGSRTFIKYPKGIPDFFKQSFPEGFTWERVTRYEDGGVVTVMQDTSLEDGCLVYHVQVRGVNFPSNGPVMQKKTKGWEPNTEMMYPADGGLRGYTHMALKVDGGGHLSCSFVTT

SEQ ID NO: 16 - protein, 672 aa, synthetic construct
  REGION 1..672: note = Recombinant sequence
MNVLSSICYGGDYNPEQWPEEIWYEDAKLMQKAGVNLVSLGIFSWSKIEPSDGVFDFEWLDKVIDILYDHGVYINLGTATATTPAWFVKKYPDSLPIDESGVIFSFGSRQHYCPNHPQLITHIKRLVRAIAERYKNHPALKMWHVNNEYACHVSKCFCENCAVAFRKWLKERYKTIDELNERWGTNFWGQRYNHWDEINPPRKAPTFINPSQELDYYRFMNDSILKLFLTEKEILRGVTPDIPVSTNFMGSFKPLNYFQWAQHIDIVTWDSYPDPREGLPIQHAMMNDLMRSLRKGQPFILMEQVTSHVNWRDINVPKPPGVMRLWSYATIARGADGIMFFQWRQSRAGAEKFHGAMVPHFLNENNRIYREVTQLGQELKKLDCLVGSRIKAEVAIIFDWENWWAVELSSKPHNKLRYIPIVEAYYRELYKRNIAVDFVRPSDDLTKYKVVIAPMLYMVKEGEDENLRQFVANGGTLIVSFFSGIVDENDRVHLGGYPGPLRDILGIFVEEFVPYPETKVNKIYSNDGEYDCTTWADIIRLEGAEPLATFKGDWYAGLPAVTRNCYGKGEGIYVGTYPDSNYLGRLLEQVFAKHHINPILEVAENVEVQQRETDEWKYLIIINHNDYEVTLSLPEDKIYQNMIDGKCFRGGELRIQGVDVAVLREHDEAGKV

SEQ ID NO: 17-19 - skipped (3 entries)

SEQ ID NO: 20 - DNA, 22 bp, synthetic construct
  misc_feature 1..22: note = Recombinant sequence
  source 1..22: mol_type = other DNA
  modified_base 6: mod_base = OTHER; note = Uracil
acaatttcacacaggaggccga

SEQ ID NO: 21 - DNA, 26 bp, synthetic construct
  misc_feature 1..26: note = Recombinant sequence
  source 1..26: mol_type = other DNA
  modified_base 7: mod_base = OTHER; note = Uracil
atcgcctcagcttactgcaggaggac

SEQ ID NO: 22 - DNA, 35 bp, synthetic construct
  misc_feature 1..35: note = Recombinant sequence
  source 1..35: mol_type = other DNA
  modified_base 7: mod_base = OTHER; note = Uracil
atcgcgtttaagacccactttcacatttaagttgt

SEQ ID NO: 23 - DNA, 34 bp, synthetic construct
  misc_feature 1..34: note = Recombinant sequence
  source 1..34: mol_type = other DNA
  modified_base 7: mod_base = OTHER; note = Uracil
atcgcctcagcttactgcaggagctcagatctgt

SEQ ID NO: 24 - DNA, 43 bp, synthetic construct
  misc_feature 1..43: note = Recombinant sequence
  source 1..43: mol_type = other DNA
  modified_base 6: mod_base = OTHER; note = Uracil
ggcgataggaggaatataccatgaacgttttatcctcaatttg

SEQ ID NO: 25 - DNA, 33 bp, synthetic construct
  misc_feature 1..33: note = Recombinant sequence
  source 1..33: mol_type = other DNA
  modified_base 6: mod_base = OTHER; note = Uracil
agagttagaaaaagaaacagaggctactctcaa

SEQ ID NO: 26 - DNA, 33 bp, synthetic construct
  misc_feature 1..33: note = Recombinant sequence
  source 1..33: mol_type = other DNA
  modified_base 7: mod_base = OTHER; note = Uracil
acgaattccctcggcaaacagagctttaaaacc

SEQ ID NO: 27 - DNA, 28 bp, synthetic construct
  misc_feature 1..28: note = Recombinant sequence
  source 1..28: mol_type = other DNA
  modified_base 8: mod_base = OTHER; note = Uracil
aacaaggtgaacatcgtgtggatacaac

SEQ ID NO: 28 - DNA, 29 bp, synthetic construct
  misc_feature 1..29: note = Recombinant sequence
  source 1..29: mol_type = other DNA
  modified_base 6: mod_base = OTHER; note = Uracil
aactctgccattatcatttccgtaatgcc

SEQ ID NO: 29 - DNA, 40 bp, synthetic construct
  misc_feature 1..40: note = Recombinant sequence
  source 1..40: mol_type = other DNA
  modified_base 10: mod_base = OTHER; note = Uracil
atccccgggtacggacttttatttcaattcgtatggtgcg

SEQ ID NO: 30 - DNA, 40 bp, synthetic construct
  misc_feature 1..40: note = Recombinant sequence
  source 1..40: mol_type = other DNA
  modified_base 6: mod_base = OTHER; note = Uracil
ggcgataggaggaatatacatggtgtctaagggcgaagag

SEQ ID NO: 31 - DNA, 26 bp, synthetic construct
  misc_feature 1..26: note = Recombinant sequence
  source 1..26: mol_type = other DNA
  modified_base 8: mod_base = OTHER; note = Uracil
ggtgcgatttacttgtacagctcgtc

SEQ ID NO: 32 - protein, 188 aa, Bacillus subtilis
MSYGDSREKILSAATRLFQLQGYYGTGLNQIIKESGAPKGSLYYHFPGGKEQLAIEAVNEMKEYIRQKIADCMEACTDPAEGIQAFLKELSCQFSCTEDIEGLPVGLLAAETSLKSEPLREACHEAYKEWASVYEEKLRQTGCSESRAKEASTVVNAMIEGGILLSLTAKNSTPLLHISSCIPDLLKR

SEQ ID NO: 33 - protein, 222 aa, Corynebacterium glutamicum
MAGAVGRPRRSAPRRAGKNPREEILDASAELFTRQGFATTSTHQIADAVGIRQASLYYHFPSKTEIFLTLLKSTVEPSTVLAEDLSTLDAGPEMRLWAIVASEVRLLLSTKWNVGRLYQLPIVGSEEFAEYHSQREALTNVFRDLATEIVGDDPRAELPFHITMSVIEMRRNDGKIPSPLSADSLPETAIMLADASLAVLGAPLPADRVEKTLELIKQADAK

SEQ ID NO: 34 - protein, 213 aa, Pseudomonas putida
MARKTAAEAEETRQRIIDAALEVFVAQGVSDATLDQIARKAGVTRGAVYWHFNGKLEVLQAVLASRQHPLELDFTPDLGIERSWEAVVVAMLDAVHSPQSKQFSEILIYQGLDESGLIHNRMVQASDRFLQYIHQVLRHAVTQGELPINLDLQTSIGVFKGLITGLLYEGLRSKDQQAQIIKVALGSFWALLREPPRFLLCEEAQIKQVKSFE

SEQ ID NO: 35 - protein, 220 aa, Streptomyces
MAQQDRAVRTRRAVLRAAAAVFAERGYAAATISEILKRAGVTKGALYFHFDSKAALAQGVLQEQLTPEYHLPRELKLQEWVDAGMTLARRLPREPFLLAGVRISADRPGREVLGSAWPAWSRLTSHVLTEAKKRGEVLPHVVPEETAQVFLGAWVGAQFVSQTLAGWEDLDDRTAALYSHLLGAIAAPPVLTRLDTAPDRGARVIAEARRRSGDLSGIAC

SEQ ID NO: 36 - protein, 188 aa, Staphylococcus aureus
MNLKDKILGVAKELFIKNGYNATTTGEIVKLSESSKGNLYYHFKTKENLFLEILNIEESKWQEQWKKEQIKCKTNREKFYLYNELSLTTEYYYPLQNAIIEFYTEYYKTNSINEKMNKLENKYIDAYHVIFKEGNLNGEWCINDVNAVSKIAANAVNGIVTFTHEQNINERIKLMNKFSQIFLNGLSK

SEQ ID NO: 37 - protein, 185 aa, Staphylococcus epidermis
MKDKIIDNAITLFSEKGYDGTTLDDISKSVNIKKASLYYHYDNKEEIYRKSVENCFNYFIDFLLRNHDDNYSIDGLYQFLFKFIFDVDERYIKLYVQLSSAPEALNSEIKHHLQEINTTLHDELIKYYDPTHIALNKEDFINLILLFLETWYFRASFSQKFGVIEDSKNRFKDQVYSLLNVFLKK

SEQ ID NO: 38 - protein, 215 aa, Streptomyces
MAKQDRAIRTRQTILDAAAQVFEKQGYQAATITEILKVAGVTKGALYFHFQSKEELALGVFDAQEPPQAVPEQPLRLQELIDMGMLFCHRLRTNVVARAGVRLSMDQQAHGLDRRGPFRRWHETLLKLLNQAKENGELLPHVVTTDSADLYVGTFAGIQVVSQTVSDYQDLEHRYALLQKHILPAIAVPSVLAALDLSEERGARLAAELAPTGKD

SEQ ID NO: 39 - protein, 268 aa, Streptomyces virginiae
MSKAAKSSRNTSPDAPESAAGNRAAAQRLKMRRELAAAAMELFASKGYEATTVDEIAAAAGVARRTFFRHFRSKEEAIFPDHDDTLIRAEAVLNAAPPHEHPLDTVCRGIKEVMKMYAASPAVSVERYRLTREVPTLREREIASVARYERLFTRYLLGHFDEHAHHDGNDDPLLAEVAASAVVTAHNHVLRRWLRAGGQGDVETQLDHAFAIVRRTFGTGIGAGRDTLPAAGPATVSAQGEVLVTVARTDAPLDEVMRTIEKALRERS

SEQ ID NO: 40 - protein, 200 aa, Pseudomonas fluorescens
MARTPSRSSIGSLRSPHTHKAILTSTIEILKECGYSGLSIESVARRAGASKPTIYRWWTNKAALIAEVYENESEQVRKFPDLGSFKADLDFLLRNLWKVWRETICGEAFRCVIAEAQLDPATLTQLKDQFMERRREMPKKLVENAISNGELPKDTNRELLLDMIFGFCWYRLLTEQLTVEQDIEEFTFLLINGVCPGTQR

SEQ ID NO: 41 - protein, 205 aa, Vibrio vulnificus
MDSIAKRPRTRLSPLKRKQQLMEIALEVFARRGIGRGGHADIAEIAQVSVATVFNYFPTREDLVDEVLNHVVRQFSNFLSDNIDLDLHAKENIANITNAMIELVVQDNHWLKVWFEWSASTRDEVWPLFVTTNRTNQLLVQNMFIKAIERGEVCDQHNPEDLANLFHGICYSLFVQANRTNNTAELSKLVSSYLDMLCIYKREHE

SEQ ID NO: 42 - protein, 213 aa, Escherichia coli
MAASASGKSKTSAGANRRRNRPSPRQRLLDSATNLFTTEGIRVIGIDRILREADVAKASLYSLFGSKDALVIAYLENLDQLWREAWRERTVGMKDPEDKIIAFFDQCIEEEPEKDFRGSHFQNAASEYPRPETDSEKGIVAAVLEHREWCHKTLTDLLTEKNGYPGTTQANQLLVFLDGGLAGSRLVHNISPLETARDLARQLLSAPPADYSI

SEQ ID NO: 43 - protein, 237 aa, Pseudomonas aeruginosa
MAQSETVERILDAAEQLFAERGFAETSLRLITSKAGVNLAAVNYHFGSKKALIQAVFSRFLGPFCASLERELERRQARPEQKPSLEELLEMLVEQALAVQPRSNNDLSIFMRLLGLAFSQSQGHLRRYLEDMYGKVFRRYMLLVNEAAPRVPPLELFWRVHFMLGAAAFSMSGIKALRAIAETDFGINTSIEQVMRLMVPFLAAGMRADSGVTDEAMAAAQLRPRSKTSTSATTAKA

SEQ ID NO: 44 - protein, 195 aa, Escherichia coli
MPKLGMQSIRRRQLIDATLEAINEVGMHDATIAQIARRAGVSTGIISHYFRDKNGLLEATMRDITSQLRDAVLNRLHALPQGSAEQRLQAIVGGNFDETQVSSAAMKAWLAFWASSMHQPMLYRLQQVSSRRLLSNLVSEFRRELPREQAQEAGYGLAALIDGLWLRAALSGKPLDKTRANSLTRHFITQHLPTD

SEQ ID NO: 45 - protein, 201 aa, Aliivibrio fischeri
MDTIQKRPRTRLSPEKRKEQLLDIAIEVFSQRGIGRGGHADIAEIAQVSVATVFNYFPTREDLVDDVLNKVENEFHQFINNSISLDLDVRSNLNTLLLNIIDSVQTGNKWIKVWFEWSTSTRDEVWPLFLSTHSNTNQVIKTMFEEGIERNEVCNDHTPENLTKMLHGICYSVFIQANRNSSSEEMEETANCFLNMLCIYK

SEQ ID NO: 46 - protein, 289 aa, Streptomyces griseus
MQQQHEVAARVRRVIDAAGVSAREFARRIVIDRRSSPGPSTAPGASPPPNWPGSRTPAAWTWDGCSGLRPVRRRRSTVRSPSAPRLPPASTEGGRPLQIVRETVRLIAERGFHAVRVADIAAACHTSTAAIHYHFPGRDELLEAAVRWCMDEDTRRRADATAGARHAGDELRLLIELQTPRTEQQRRQWCVWLDLWAEAARSTTVGQLHVEYYRQWRGTVADVIRRGVGQGVFRPVDADGAALTLTALIDGLASQVLATAPGHPGTGAQTMHDALIAHVSACLAAPAAD

SEQ ID NO: 47 - protein, 203 aa, Vibrio cholerae
MDASIEKRPRTRLSPQKRKLQLMEIALEVFAKRGIGRGGHADIAEIAQVSVATVFNYFPTREDLVDDVLNFVVRQYSNFLTDHIDLDLDVKTNLQTVCKEMVKLAMTDCHWLKVWFEWSASTRDEVWPLFVSTNRTNQLLIRNMFMKAMERGELCEKHDVDNMASLFHGIFYSIFLQVNRLGEQEAVYKLADSYLNMLCIYKN

SEQ ID NO: 48 - protein, 211 aa, Bacillus cereus
MKYILFEVCEMGKSREQTMENILKAAKKKFGERGYEGTSIQEIAKEAKVNVAMASYYFNGKENLYYEVFKKYGLANELPNFLEKNQFNPINALREYLTVFTTHIKENPEIGTLAYEEIIKESARLEKIKPYFIGSFEQLKEILQEGEKQGVFHFFSINHTIHWITSIVLFPKFKKFIDSLGPNETNDTNHEWMPEDLVSRIISALTDKPNI

SEQ ID NO: 49 - protein, 219 aa, Agrobacterium fabrum
MNKTIDQVRKGDRKSDLPVRRRPRRSAEETRRDILAKAEELFRERGFNAVAIADIASALNMSPANVFKHFSSKNALVDAIGFGQIGVFERQICPLDKSHAPLDRLRHLARNLMEQHHQDLNKNPYVFEMILMTAKQDMKCGDYYKSVIAKLLAEIIRDGVEAGLYIATDIPVLAETVLHALTSVIHPVLIAQEDIGNLATRCDQLVDLIDAGLRNPLAK

SEQ ID NO: 50 - protein, 192 aa, Bacillus megaterium
MESTPTKQKAIFSASLLLFAERGFDATTMPMIAENAKVGAGTIYRYFKNKESLVNELFQQHVNEFLQCIESGLANERDGYRDGFHHIFEGMVTFTKNHPRALGFIKTHSQGTFLTEESRLAYQKLVEFVCTFFREGQKQGVIRNLPENALIAILFGSFMEVYEMIENDYLSLTDELLTGVEESLWAALSRQS

SEQ ID NO: 51 - protein, 191 aa, Thermus thermophilus
MVTTTRDRILEEAAKLFTEKGYEATSVQDLAQALGLSKAALYHHFGSKEEILYEISLLALKGLVAAGEKALEVADPKEALRRFMEAHARYFEENYPFFVTMLQGIKSLSPENRLKTIALRDRHEENLRAILRRGVEQGVFREVDVALAGRAVLSMLNWMIRWFRPDGPMRAEEVARAYHDLILRGLERGSA

SEQ ID NO: 52 - protein, 203 aa, Thermus thermophilus
MDRRSQILTIAGHLFSQRGYHATSMRELARHLNLQGGSLYAHIQSKEELLLEVVRQAAERFQKVLEELPSGDPVTRMKALVKGHLRVIAEELPRATVFFHEWKHLSPPLLEEAKALRRRYEEGVQAVVEEGVRAGVFRVENVRLATLFVLSALNWTYQWYRPDGPLSLEALAEAYAELVLKALGVAASPEGERREEGGEDGEA

SEQ ID NO: 53 - protein, 205 aa, Thermus thermophilus
MAVREYQKKRRRERIFRAAMELFRNRGFQETTATEIAKAAHVSRGTFFNYYPYKEAVLLDYGSQLLAGLREEVRRLLAQGREPVEVLRHLFRVLAEGTAREKDLLLPMFYELLNPDPVRARAAFEALPLGDLIAEILKPLREQGVLRQDFSLERMGRTLADLYFLSALRWAAYTPGRDLAEELEKNLRLLLEGMLVREAPAPGGL

SEQ ID NO: 54 - protein, 206 aa, Colwellia psychrerythraea
MVKDSTVDIKKLSPRGLLILDAAQNLFYNLGFDETSLAMIIKDAGGSRRSIYNEFGNKHGLLMAVIQRQVKTQSEILTSINRDLEAKEALNQVCFKFVVGMLSPELMSLFRLVVQQVVKFPELGEMIYKNGPMTGILPLVDYLTWLTEQKILKIDNCHFSAQMLMEMAKGPLHTRSLLLPDKQVSDEEITYQVTKAVDLFLHAHKM

SEQ ID NO: 55 - protein, 199 aa, Sporosarcina psychrophila
MENNKTISSKRQNRTKEHFKLALIELIKRNGYHSVSVKDIVNQASYNRSTFYVHYQDKIQLAEDLLESMLKGLENSVGKPYIPGQKFYTTKLNTPSFNIISYIYENRNFFLIKYDEPLPGLHTRFPQTILKIYQEQFNFQTINNIPVNMDYFKRYTAFGFYGLILNWINSDFKESQEEFIEEVIALTKTHIYSIEYIGE

SEQ ID NO: 56 - protein, 215 aa, Moritella marina
MVSKLDTKTRILDAAESLFAEHGFSDTSLRLITTRASVNLASVNYHFGSKKELIQAVIARHLELFMPLLNEKLTALCTQVEKPSLLDVFNSFVDPLLALEQQSKNGTVIFMQLLGRSYTDEQGHLRWYTTTHYGEVLANITKALLKANPALSSQELFWRLHFTLGTAVFTMGSSGALMDIAKADFNQDIDVEGLIRKVIPYLASGMNTSTETTSM

SEQ ID NO: 57 - protein, 61 aa, Escherichia coli
RLNRESVIDAALELLNETGIDGLTTRKLAQKLGIEQPTLYWHVKNKRALLDALAVEILARH

SEQ ID NO: 58 - protein, 61 aa, Bacillus subtilis
GDSREKILHTASRLFQLQGYHATGLNQIVKESGAPKGSLYHFFPNGKEELAIEAVTYTGKI

SEQ ID NO: 59 - protein, 61 aa, Corynebacterium glutamicum
KNPREEILDASAELFTRQGFATTSTHQIADAVGIRQASLYYHFPSKTEIFLTLLKSTVEPS

SEQ ID NO: 60 - protein, 61 aa, Pseudomonas putida
EETRQRIIDAALEVFVAQGVSDATLDQIARKAGVTRGAVYWHFNGKLEVLQAVLASRQHPL

SEQ ID NO: 61 - protein, 61 aa, Staphylococcus aureus
MNLKDKILGVAKELFIKNGYNATTTGEIVKLSESSKGNLYYHFKTKENLFLEILNIEESKW

SEQ ID NO: 62 - protein, 59 aa, Staphylococcus epidermis
MKDKIIDNAITLFSEKGYDGTTLDDISKSVNIKKASLYYHYDNKEEIYRKSVENCFNYF

SEQ ID NO: 63 - protein, 61 aa, Pseudomonas fluorescens
PHTHKAILTSTIEILKECGYSGLSIESVARRAGASKPTIYRWWTNKAALIAEVYENESEQV

SEQ ID NO: 64 - protein, 61 aa, Vibrio vulnificus
LKRKQQLMEIALEVFARRGIGRGGHADIAEIAQVSVATVFNYFPTREDLVDEVLNHVVRQF

SEQ ID NO: 65 - protein, 68 aa, Escherichia coli
VSLTLQVENDLKHQLSIGALKPGARLITKNLAEQLGMSITPVREALLRLVSVNALSVAPAQAFTVPEV

SEQ ID NO: 66 - protein, 61 aa, Pseudomonas aeruginosa
SETVERILDAAEQLFAEKGFAETSLRLITSKAGVNLAAVNYHFGSKKALIQAVFSRFLGPF

SEQ ID NO: 67 - protein, 61 aa, Escherichia coli
SIRRRQLIDATLEAINEVGMHDATIAQIARRAGVSTGIISHYFRDKNGLLEATMRDITSQL

SEQ ID NO: 68 - protein, 61 aa, Aliivibrio fischeri
EKRKEQLLDIAIEVFSQRGIGRGGHADIAEIAQVSVATVFNYFPTREDLVDDVLNKVENEF

SEQ ID NO: 69 - protein, 61 aa, Vibrio cholerae
QKRKLQLMEIALEVFAKRGIGRGGHADIAEIAQVSVATVFNYFPTREDLVDDVLNFVVRQY

SEQ ID NO: 70 - protein, 61 aa, Bacillus cereus
EQTMENILKAAKKKFGERGYEGTSIQEIAKEAKVNVAMASYYFNGKENLYYEVFKKYGLAN

SEQ ID NO: 71 - protein, 61 aa, Agrobacterium fabrum
EETRRDILAKAEELFRERGFNAVAIADIASALNMSPANVFKHFSSKNALVDAIGFGQIGVF

SEQ ID NO: 72 - protein, 61 aa, Bacillus megaterium
PTKQKAIFSASLLLFAERGFDATTMPMIAENAKVGAGTIYRYFKNKESLVNELFQQHVNEF

SEQ ID NO: 73 - protein, 61 aa, Thermus thermophilus
TTTRDRILEEAAKLFTEKGYEATSVQDLAQALGLSKAALYHHFGSKEEILYEISLLALKGL

SEQ ID NO: 74 - protein, 61 aa, Thermus thermophilus
MDRRSQILTIAGHLFSQRGYHATSMRELARHLNLQGGSLYAHIQSKEELLLEVVRQAAERF

SEQ ID NO: 75 - protein, 61 aa, Thermus thermophilus
KRRRERIFRAAMELFRNRGFQETTATEIAKAAHVSRGTFFNYYPYKEAVLLDYGSQLLAGL

SEQ ID NO: 76 - protein, 61 aa, Colwellia psychrerythraea
SPRGLLILDAAQNLFYNLGFDETSLAMIIKDAGGSRRSIYNEFGNKHGLLMAVIQRQVKTQ

SEQ ID NO: 77 - protein, 61 aa, Sporosarcina psychrophila
NRTKEHFKLALIELIKRNGYHSVSVKDIVNQASYNRSTFYVHYQDKIQLAEDLLESMLKGL

SEQ ID NO: 78 - protein, 61 aa, Moritella marina
LDTKTRILDAAESLFAEHGFSDTSLRLITTRASVNLASVNYHFGSKKELIQAVIARHLELF

SEQ ID NO: 79 - protein, 207 aa, Providencia stuartii
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCENGS

SEQ ID NO: 80 - protein, 207 aa, Enterobacter roggenkampii
MSRLDKSKVINSALELLNEVGIEGLTTRKLAQKLGVEQPTLYWHVKNKRALLDALAIEMLDRHHTHFCPLEGESWRDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 81 - protein, 199 aa, Shigella flexneri
MINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 82 - protein, 149 aa, Acinetobacter baumannii
MLDRHHTHFCPLEGESWQDFLRNNAKSFRCALLSHRDGAKVHLSTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS